(19) United States
(12) Patent Application Publication
(10) Pub. No.: US 2026/0167991 A1
(43) Pub. Date: Jun. 18, 2026

(54) LIVER DETARGETED CAPSIDS

(71) Applicant: KATE THERAPEUTICS, INC., San Diego, CA (US)

(72) Inventors: Sharif TABEBORDBAR, San Diego, CA (US); Saurav SESHADRI, San Diego, CA (US); Simon YE, San Diego, CA (US)

(21) Appl. No.: 19/406,703

(22) Filed: Dec. 2, 2025

Related U.S. Application Data
(63) Continuation of application No. PCT/US2024/044341, filed on Aug. 29, 2024.
(60) Provisional application No. 63/585,343, filed on Sep. 26, 2023.
(60) Provisional application No. 63/644,785, filed on May 9, 2024.

(51) Int. Cl. C12N 15/86

(52) U.S. Cl. CPC C12N 15/86; C12N 2750/14143

(57) ABSTRACT

The present invention provides novel capsid variants for viral vectors that detarget liver tissue and effectively transduce muscle tissue at the same time.

### RELATED APPLICATIONS

This application is a continuation of International Application No. PCT/US2024/044341, filed Aug. 29, 2024, which claims the benefit of U.S. Provisional Application No. 63/644,785, filed May 9, 2024, and U.S. Provisional Application No. 63/585,343, filed Sep. 26, 2023, the entire contents of each of which are incorporated herein by reference in their entirety.

### FIELD OF DISCLOSURE

This disclosure relates to viral capsids.

### SEQUENCE LISTING

The instant application contains a Sequence Listing which has been submitted electronically in XML format and is hereby incorporated by reference in its entirety. Said XML copy, created on Dec. 1, 2025, is named KATE-024-01WO-ST26.XML and is 3,141,632 bytes in size.

### BACKGROUND

Recombinant AAVs (rAAVs) are the most commonly used delivery vehicles for gene therapy and gene editing. Nonetheless, rAAVs that contain natural capsid variants have limited cell tropism. Indeed, rAAVs used today mainly infect the liver after systemic delivery. Further, the transduction efficiency of conventional rAAVs in other cell-types, tissues, and organs by these conventional rAAVs with natural capsid variants is limited. Therefore, AAV-mediated polynucleotide delivery for diseased that affect cells, tissues, and organs other than the liver, such as the central nervous system) typically requires an injection of a large dose of virus (typically about 2×10¹⁴ vg/kg), which often results in liver toxicity. Furthermore, because large doses are required when using conventional rAAVs, manufacturing sufficient amounts of a therapeutic rAAV needed to dose adult patients is extremely challenging. Additionally, due to differences in gene expression and physiology, mouse and primate models respond differently to viral capsids. Transduction efficiency of different virus particles varies between different species, and as a result, preclinical studies in mice often do not accurately reflect results in primates, including humans. As such there exists a need for improved rAAVs for use in the treatment of various genetic diseases.

### SUMMARY

The present invention provides novel capsid protein variants for viral vectors that detarget liver tissue and target skeletal muscle and heart tissue at the same time. Aspects of the present invention provide adeno-associated virus (AAV) vector's comprising a capsid protein comprising the amino acid sequence RGDR. In the capsid protein, RGDR may be inserted after amino acid 455 in reference to an AAV9 capsid or equivalent position in another AAV capsid. AAV vectors may comprise the amino acid sequence X₁NX₂X₃X₄RGDRX₅X₆L, wherein X₁, X₂, X₃, X₄, X₅, and X₆ may be any amino acid.

In aspects of the invention, X₁ may be an amino acid selected from the group consisting of: A, I, F, G, H, L, M, Q, S, T, V. In preferred aspects of the invention, X₁ may be an amino acid selected from the group consisting of: A, I, L, M, S, V.

In aspects of the invention, X₂ may be an amino acid selected from the group consisting of A, G, S, T, Y.

In aspects of the invention, X₃ may be an amino acid selected from the group consisting of S, N, G, P. In preferred aspects of the invention, X₃ may be S.

In aspects of the invention, X₄ may be an amino acid selected from the group consisting of A, G, H, I, M, S, T, V.

In aspects of the invention, X₅ may be an amino acid selected from the group consisting of A, G, Q.

In aspects of the invention, X₆ may be an amino acid selected from the group consisting of A, I, L, M, N, Y. In preferred aspects of the invention, X₆ may be selected from the group consisting of A, S, and Y.

X₁ may is located at amino acid 451, X₂ located at amino acid 453, X₃ located at amino acid 454, X₄ is located amino acid 455, and RGDRX₅X₆L inserted after amino acid 455 in reference to an AAV9 capsid or equivalent position in another AAV capsid.

In aspects of the invention, two amino acids from X₁, X₂, X₃, and X₄ are wild type amino acids in reference to an AAV9 capsid or equivalent position in another AAV capsid and two amino acids from X₁, X₂, X₃, and X₄ are not wild type amino acids.

For example, capsid proteins variants of the invention may comprise a sequence as set forth in Table 1a. Notably, capsid protein variants on the invention comprise deletions, substitutions, and/or insertions relative to wild-type viral vector capsids.

In aspects of the invention, the capsid protein comprises an amino acid sequence selected from Table 1a and the amino acid sequence is in hypervariable region IV (HVR IV) relative to wild-type AAV9. The capsid protein variant may comprise substitutions at amino acids 451-455 relative to a wild-type AAV9 vector capsid. For example, the substitutions at amino acids 451-455 relative to a wild-type AAV9 vector capsid may be an amino acid sequence selected from column 1 of Table 1b. In aspects of the invention, the capsid protein variant may further comprise an insert. For example, the capsid protein may comprise a 7-mer insert selected from column 2 of Table 1b. The insert may be in the location after amino acid 455 relative to a wild-type AAV9 vector.

Advantageously, viral vectors comprising an amino acid sequence of the invention exhibit muscle tropism as compared to a wild-type AAV vector.

In aspects of the invention, the capsid protein further comprises a deletion of G267 in reference to an AAV9 capsid or equivalent position in another AAV capsid. Advantageously, the vector may exhibit reduced liver tropism as compared to a wild-type AAV vector.

As described, for the HVR IV variants, the 5 amino acids upstream are at positions 451-455, shown in column 1 of Table 1b. The 7-mer insert for HVR IV variants starts with “RGD” and is inserted after amino acid 455, shown in column 2 of Table 1b.

TABLE 1a
HVR IV Capsid Variants

| Amino Acid | |
|---|---|
| Sequence | SEQ ID NO: |
| ANASARGDRAAL | 2 |
| ANASARGDRGSL | 3 |
| ANASARGDRGYL | 4 |
| ANASARGDRQAL | 5 |
| ANASARGDRQSL | 6 |
| ANASARGDRQYL | 7 |
| ANASGRGDRAAL | 8 |
| ANASGRGDRASL | 9 |
| ANASGRGDRAYL | 10 |
| ANASGRGDRGAL | 11 |
| ANASGRGDRGSL | 12 |
| ANASGRGDRGYL | 13 |
| ANASGRGDRQAL | 14 |
| ANASGRGDRQSL | 15 |
| ANASGRGDRQYL | 16 |
| ANASMRGDRAAL | 17 |
| ANASMRGDRASL | 18 |
| ANASMRGDRAYL | 19 |
| ANASMRGDRQAL | 20 |
| ANASMRGDRQSL | 21 |
| ANASSRGDRGSL | 22 |
| ANASSRGDRGYL | 23 |
| ANASSRGDRQAL | 24 |
| ANASSRGDRQSL | 25 |
| ANASSRGDRQYL | 26 |
| ANASTRGDRASL | 27 |
| ANASTRGDRAYL | 28 |
| ANASTRGDRGAL | 29 |
| ANASTRGDRGSL | 30 |
| ANASTRGDRGYL | 31 |
| ANASTRGDRQAL | 32 |
| ANASTRGDRQSL | 33 |
| ANASTRGDRQYL | 34 |
| ANASVRGDRASL | 35 |
| ANASVRGDRGYL | 36 |
| ANASVRGDRQAL | 37 |
| ANASVRGDRQSL | 38 |
| ANASVRGDRQYL | 39 |
| ANGNFRGDRQNL | 40 |
| ANGNVRGDRGML | 41 |
| ANGSARGDRAYL | 42 |
| ANGSARGDRGSL | 43 |
| ANGSARGDRGYL | 44 |
| ANGSARGDRQAL | 45 |
| ANGSARGDRQYL | 46 |
| ANGSGRGDRAAL | 47 |
| ANGSGRGDRAYL | 48 |
| ANGSGRGDRGAL | 49 |
| ANGSGRGDRGSL | 50 |
| ANGSGRGDRGYL | 51 |
| ANGSGRGDRQAL | 52 |
| ANGSGRGDRQSL | 53 |
| ANGSGRGDRQYL | 54 |
| ANGSMRGDRGYL | 55 |
| ANGSMRGDRQAL | 56 |
| ANGSMRGDRQSL | 57 |
| ANGSMRGDRQYL | 58 |
| ANGSSRGDRAYL | 59 |
| ANGSSRGDRGSL | 60 |
| ANGSSRGDRGYL | 61 |
| ANGSSRGDRQAL | 62 |
| ANGSSRGDRQSL | 63 |
| ANGSSRGDRQYL | 64 |
| ANGSTRGDRAAL | 65 |
| ANGSTRGDRAYL | 66 |
| ANGSTRGDRGAL | 67 |
| ANGSTRGDRGYL | 68 |
| ANGSTRGDRQAL | 69 |
| ANGSTRGDRQSL | 70 |
| ANGSTRGDRQYL | 71 |
| ANGSVRGDRAYL | 72 |
| ANGSVRGDRGYL | 73 |
| ANGSVRGDRQAL | 74 |
| ANGSVRGDRQYL | 75 |
| ANSGMRGDRGAL | 76 |
| ANSSARGDRGAL | 77 |
| ANSSARGDRGSL | 78 |
| ANSSARGDRGYL | 79 |
| ANSSARGDRQAL | 80 |
| ANSSARGDRQSL | 81 |
| ANSSARGDRQYL | 82 |
| ANSSGRGDRAAL | 83 |
| ANSSGRGDRASL | 84 |
| ANSSGRGDRAYL | 85 |
| ANSSGRGDRGAL | 86 |
| ANSSGRGDRGSL | 87 |
| ANSSGRGDRGYL | 88 |
| ANSSGRGDRQAL | 89 |
| ANSSGRGDRQSL | 90 |
| ANSSGRGDRQYL | 91 |
| ANSSMRGDRAYL | 92 |
| ANSSMRGDRQAL | 93 |
| ANSSMRGDRQSL | 94 |
| ANSSMRGDRQYL | 95 |
| ANSSSRGDRGSL | 96 |
| ANSSSRGDRGYL | 97 |
| ANSSSRGDRQAL | 98 |
| ANSSSRGDRQSL | 99 |
| ANSSSRGDRQYL | 100 |
| ANSSTRGDRAAL | 101 |
| ANSSTRGDRGSL | 102 |
| ANSSTRGDRQAL | 103 |
| ANSSTRGDRQSL | 104 |
| ANSSTRGDRQYL | 105 |
| ANSSVRGDRAYL | 106 |
| ANSSVRGDRGYL | 107 |
| ANSSVRGDRQAL | 108 |
| ANSSVRGDRQSL | 109 |
| ANSSVRGDRQYL | 110 |
| ANTSARGDRAYL | 111 |
| ANTSARGDRGAL | 112 |
| ANTSARGDRGSL | 113 |
| ANTSARGDRGYL | 114 |
| ANTSARGDRQAL | 115 |
| ANTSARGDRQYL | 116 |
| ANTSGRGDRASL | 117 |
| ANTSGRGDRGAL | 118 |
| ANTSGRGDRGSL | 119 |
| ANTSGRGDRGYL | 120 |
| ANTSGRGDRQAL | 121 |
| ANTSGRGDRQSL | 122 |
| ANTSGRGDRQYL | 123 |
| ANTSMRGDRAYL | 124 |
| ANTSMRGDRQAL | 125 |
| ANTSMRGDRQSL | 126 |
| ANTSMRGDRQYL | 127 |
| ANTSSRGDRGSL | 128 |
| ANTSSRGDRQAL | 129 |
| ANTSSRGDRQYL | 130 |
| ANTSTRGDRAAL | 131 |
| ANTSTRGDRQAL | 132 |
| ANTSTRGDRQSL | 133 |
| ANTSTRGDRQYL | 134 |
| ANTSVRGDRAYL | 135 |
| ANTSVRGDRGSL | 136 |
| ANTSVRGDRQAL | 137 |
| ANTSVRGDRQSL | 138 |
| ANTSVRGDRQYL | 139 |
| ANYPHRGDRGAL | 140 |
| ANYSARGDRAAL | 141 |
| ANYSARGDRASL | 142 |
| ANYSARGDRGAL | 143 |
| ANYSARGDRGSL | 144 |
| ANYSARGDRQAL | 145 |
| ANYSARGDRQSL | 146 |
| ANYSGRGDRAAL | 147 |
| ANYSGRGDRASL | 148 |
| ANYSGRGDRGAL | 149 |
| ANYSGRGDRGSL | 150 |
| ANYSGRGDRQAL | 151 |
| ANYSGRGDRQSL | 152 |
| ANYSGRGDRQYL | 153 |
| ANYSMRGDRAAL | 154 |
| ANYSMRGDRASL | 155 |
| ANYSMRGDRAYL | 156 |
| ANYSMRGDRQAL | 157 |
| ANYSMRGDRQSL | 158 |
| ANYSSRGDRGAL | 159 |
| ANYSSRGDRGSL | 160 |
| ANYSSRGDRQAL | 161 |
| ANYSSRGDRQSL | 162 |
| ANYSSRGDRQYL | 163 |
| ANYSTRGDRQAL | 164 |
| ANYSTRGDRQSL | 165 |
| ANYSTRGDRQYL | 166 |
| ANYSVRGDRQAL | 167 |
| ANYSVRGDRQSL | 168 |
| ANYSVRGDRQYL | 169 |
| FNSGMRGDRGAL | 170 |
| GNSGMRGDRGAL | 171 |
| HNSGMRGDRGAL | 172 |
| LNASARGDRAAL | 173 |
| LNASARGDRASL | 174 |
| LNASARGDRAYL | 175 |
| LNASARGDRGAL | 176 |
| LNASARGDRGSL | 177 |
| LNASARGDRGYL | 178 |
| LNASARGDRQAL | 179 |
| LNASARGDRQSL | 180 |
| LNASARGDRQYL | 181 |
| LNASGRGDRAAL | 182 |
| LNASGRGDRASL | 183 |
| LNASGRGDRAYL | 184 |
| LNASGRGDRGAL | 185 |
| LNASGRGDRGSL | 186 |
| LNASGRGDRGYL | 187 |
| LNASGRGDRQAL | 188 |
| LNASGRGDRQSL | 189 |
| LNASGRGDRQYL | 190 |
| LNASIRGDRQLL | 191 |
| LNASMRGDRAAL | 192 |
| LNASMRGDRASL | 193 |
| LNASMRGDRAYL | 194 |
| LNASMRGDRGAL | 195 |
| LNASMRGDRGSL | 196 |
| LNASMRGDRGYL | 197 |
| LNASMRGDRQAL | 198 |
| LNASMRGDRQSL | 199 |
| LNASMRGDRQYL | 200 |
| LNASSRGDRAAL | 201 |
| LNASSRGDRASL | 202 |
| LNASSRGDRGAL | 203 |
| LNASSRGDRGSL | 204 |
| LNASSRGDRQAL | 205 |
| LNASSRGDRQSL | 206 |
| LNASSRGDRQYL | 207 |
| LNASTRGDRAAL | 208 |
| LNASTRGDRASL | 209 |
| LNASTRGDRGAL | 210 |
| LNASTRGDRGSL | 211 |
| LNASTRGDRGYL | 212 |
| LNASTRGDRQAL | 213 |
| LNASTRGDRQSL | 214 |
| LNASTRGDRQYL | 215 |
| LNASVRGDRAAL | 216 |
| LNASVRGDRASL | 217 |
| LNASVRGDRGAL | 218 |
| LNASVRGDRGSL | 219 |
| LNASVRGDRQAL | 220 |
| LNASVRGDRQSL | 221 |
| LNASVRGDRQYL | 222 |
| LNGNFRGDRQNL | 223 |
| LNGNVRGDRGML | 224 |
| LNGSARGDRAAL | 225 |
| LNGSARGDRAYL | 226 |
| LNGSARGDRGSL | 227 |
| LNGSARGDRGYL | 228 |
| LNGSARGDRQAL | 229 |
| LNGSARGDRQSL | 230 |
| LNGSARGDRQYL | 231 |
| LNGSGRGDRAAL | 232 |
| LNGSGRGDRAYL | 233 |
| LNGSGRGDRGAL | 234 |
| LNGSGRGDRGSL | 235 |
| LNGSGRGDRGYL | 236 |
| LNGSGRGDRQAL | 237 |
| LNGSGRGDRQYL | 238 |
| LNGSMRGDRASL | 239 |
| LNGSMRGDRGYL | 240 |
| LNGSMRGDRQAL | 241 |
| LNGSMRGDRQSL | 242 |
| LNGSMRGDRQYL | 243 |
| LNGSSRGDRAAL | 244 |
| LNGSSRGDRAYL | 245 |
| LNGSSRGDRGAL | 246 |
| LNGSSRGDRQYL | 247 |
| LNGSTRGDRAAL | 248 |
| LNGSTRGDRASL | 249 |
| LNGSTRGDRAYL | 250 |
| LNGSTRGDRGAL | 251 |
| LNGSTRGDRGSL | 252 |
| LNGSTRGDRGYL | 253 |
| LNGSTRGDRQAL | 254 |
| LNGSTRGDRQSL | 255 |
| LNGSTRGDRQYL | 256 |
| LNGSVRGDRASL | 257 |
| LNGSVRGDRAYL | 258 |
| LNGSVRGDRGYL | 259 |
| LNGSVRGDRQAL | 260 |
| LNGSVRGDRQSL | 261 |
| LNGSVRGDRQYL | 262 |
| LNSGMRGDRGAL | 263 |
| LNSSARGDRASL | 264 |
| LNSSARGDRAYL | 265 |
| LNSSARGDRGAL | 266 |
| LNSSARGDRGIL | 267 |
| LNSSARGDRGSL | 268 |
| LNSSARGDRGYL | 269 |
| LNSSARGDRQAL | 270 |
| LNSSARGDRQSL | 271 |
| LNSSARGDRQYL | 272 |
| LNSSGRGDRAAL | 273 |
| LNSSGRGDRASL | 274 |
| LNSSGRGDRAYL | 275 |
| LNSSGRGDRGAL | 276 |
| LNSSGRGDRGSL | 277 |
| LNSSGRGDRGYL | 278 |
| LNSSGRGDRQAL | 279 |
| LNSSGRGDRQSL | 280 |
| LNSSGRGDRQYL | 281 |
| LNSSMRGDRAAL | 282 |
| LNSSMRGDRASL | 283 |
| LNSSMRGDRAYL | 284 |
| LNSSMRGDRGAL | 285 |
| LNSSMRGDRGSL | 286 |
| LNSSMRGDRQAL | 287 |
| LNSSMRGDRQSL | 288 |
| LNSSMRGDRQYL | 289 |
| LNSSSRGDRAAL | 290 |
| LNSSSRGDRGAL | 291 |
| LNSSSRGDRGSL | 292 |
| LNSSSRGDRQAL | 293 |
| LNSSSRGDRQSL | 294 |
| LNSSSRGDRQYL | 295 |
| LNSSTRGDRAAL | 296 |
| LNSSTRGDRASL | 297 |
| LNSSTRGDRGAL | 298 |
| LNSSTRGDRGSL | 299 |
| LNSSTRGDRQAL | 300 |
| LNSSTRGDRQSL | 301 |
| LNSSTRGDRQYL | 302 |
| LNSSVRGDRAAL | 303 |
| LNSSVRGDRASL | 304 |
| LNSSVRGDRGAL | 305 |
| LNSSVRGDRGSL | 306 |
| LNSSVRGDRGYL | 307 |
| LNSSVRGDRQAL | 308 |
| LNSSVRGDRQSL | 309 |
| LNSSVRGDRQYL | 310 |
| LNTSARGDRAAL | 311 |
| LNTSARGDRASL | 312 |
| LNTSARGDRAYL | 313 |
| LNTSARGDRGAL | 314 |
| LNTSARGDRGSL | 315 |
| LNTSARGDRGYL | 316 |
| LNTSARGDRQAL | 317 |
| LNTSARGDRQSL | 318 |
| LNTSARGDRQYL | 319 |
| LNTSGRGDRAAL | 320 |
| LNTSGRGDRASL | 321 |
| LNTSGRGDRAYL | 322 |
| LNTSGRGDRGAL | 323 |
| LNTSGRGDRGSL | 324 |
| LNTSGRGDRGYL | 325 |
| LNTSGRGDRQAL | 326 |
| LNTSGRGDRQSL | 327 |
| LNTSGRGDRQYL | 328 |
| LNTSMRGDRAAL | 329 |
| LNTSMRGDRASL | 330 |
| LNTSMRGDRAYL | 331 |
| LNTSMRGDRGAL | 332 |
| LNTSMRGDRGSL | 333 |
| LNTSMRGDRQAL | 334 |
| LNTSMRGDRQSL | 335 |
| LNTSMRGDRQYL | 336 |
| LNTSSRGDRAAL | 337 |
| LNTSSRGDRGSL | 338 |
| LNTSSRGDRQAL | 339 |
| LNTSSRGDRQSL | 340 |
| LNTSSRGDRQYL | 341 |
| LNTSTRGDRAAL | 342 |
| LNTSTRGDRASL | 343 |
| LNTSTRGDRGAL | 344 |
| LNTSTRGDRGSL | 345 |
| LNTSTRGDRQAL | 346 |
| LNTSTRGDRQSL | 347 |
| LNTSTRGDRQYL | 348 |
| LNTSVRGDRAAL | 349 |
| LNTSVRGDRASL | 350 |
| LNTSVRGDRAYL | 351 |
| LNTSVRGDRGAL | 352 |
| LNTSVRGDRGSL | 353 |
| LNTSVRGDRQAL | 354 |
| LNTSVRGDRQSL | 355 |
| LNYPHRGDRGAL | 356 |
| LNYSARGDRGAL | 357 |
| LNYSARGDRGSL | 358 |
| LNYSARGDRQAL | 359 |
| LNYSARGDRQSL | 360 |
| LNYSARGDRQYL | 361 |
| LNYSGRGDRAAL | 362 |
| LNYSGRGDRASL | 363 |
| LNYSGRGDRGAL | 364 |
| LNYSGRGDRGSL | 365 |
| LNYSGRGDRQAL | 366 |
| LNYSGRGDRQSL | 367 |
| LNYSGRGDRQYL | 368 |
| LNYSMRGDRQAL | 369 |
| LNYSMRGDRQSL | 370 |
| LNYSSRGDRGAL | 371 |
| LNYSSRGDRGSL | 372 |
| LNYSSRGDRQAL | 373 |
| LNYSSRGDRQSL | 374 |
| LNYSSRGDRQYL | 375 |
| LNYSTRGDRASL | 376 |
| LNYSTRGDRGAL | 377 |
| LNYSTRGDRGSL | 378 |
| LNYSTRGDRQAL | 379 |
| LNYSTRGDRQSL | 380 |
| LNYSTRGDRQYL | 381 |
| LNYSVRGDRQAL | 382 |
| LNYSVRGDRQSL | 383 |
| MNASARGDRAAL | 384 |
| MNASARGDRAYL | 385 |
| MNASARGDRGSL | 386 |
| MNASARGDRGYL | 387 |
| MNASARGDRQAL | 388 |
| MNASARGDRQSL | 389 |
| MNASARGDRQYL | 390 |
| MNASGRGDRAAL | 391 |
| MNASGRGDRASL | 392 |
| MNASGRGDRAYL | 393 |
| MNASGRGDRGSL | 394 |
| MNASGRGDRQAL | 395 |
| MNASGRGDRQSL | 396 |
| MNASGRGDRQYL | 397 |
| MNASIRGDRQLL | 398 |
| MNASMRGDRAAL | 399 |
| MNASMRGDRAYL | 400 |
| MNASMRGDRGAL | 401 |
| MNASMRGDRQAL | 402 |
| MNASMRGDRQSL | 403 |
| MNASSRGDRAAL | 404 |
| MNASTRGDRAAL | 405 |
| MNASTRGDRASL | 406 |
| MNASTRGDRAYL | 407 |
| MNASTRGDRGAL | 408 |
| MNASTRGDRGSL | 409 |
| MNASTRGDRQAL | 410 |
| MNASTRGDRQSL | 411 |
| MNASTRGDRQYL | 412 |
| MNASVRGDRAAL | 413 |
| MNASVRGDRASL | 414 |
| MNASVRGDRAYL | 415 |
| MNASVRGDRQAL | 416 |
| MNASVRGDRQSL | 417 |
| MNASVRGDRQYL | 418 |
| MNGNFRGDRQNL | 419 |
| MNGSARGDRAYL | 420 |
| MNGSARGDRGYL | 421 |
| MNGSARGDRQAL | 422 |
| MNGSARGDRQSL | 423 |
| MNGSARGDRQYL | 424 |
| MNGSGRGDRAAL | 425 |
| MNGSGRGDRAYL | 426 |
| MNGSGRGDRGAL | 427 |
| MNGSGRGDRGSL | 428 |
| MNGSGRGDRGYL | 429 |
| MNGSGRGDRQAL | 430 |
| MNGSGRGDRQSL | 431 |
| MNGSGRGDRQYL | 432 |
| MNGSMRGDRQAL | 433 |
| MNGSMRGDRQSL | 434 |
| MNGSMRGDRQYL | 435 |
| MNGSSRGDRQYL | 436 |
| MNGSTRGDRAYL | 437 |
| MNGSTRGDRGAL | 438 |
| MNGSTRGDRGYL | 439 |
| MNGSTRGDRQAL | 440 |
| MNGSTRGDRQSL | 441 |
| MNGSTRGDRQYL | 442 |
| MNGSVRGDRGYL | 443 |
| MNGSVRGDRQAL | 444 |
| MNGSVRGDRQYL | 445 |
| MNSGMRGDRGAL | 446 |
| MNSSARGDRAAL | 447 |
| MNSSARGDRAYL | 448 |
| MNSSARGDRGSL | 449 |
| MNSSARGDRGYL | 450 |
| MNSSARGDRQAL | 451 |
| MNSSARGDRQSL | 452 |
| MNSSARGDRQYL | 453 |
| MNSSGRGDRAAL | 454 |
| MNSSGRGDRASL | 455 |
| MNSSGRGDRAYL | 456 |
| MNSSGRGDRGAL | 457 |
| MNSSGRGDRGSL | 458 |
| MNSSGRGDRQAL | 459 |
| MNSSGRGDRQSL | 460 |
| MNSSGRGDRQYL | 461 |
| MNSSMRGDRAAL | 462 |
| MNSSMRGDRASL | 463 |
| MNSSMRGDRAYL | 464 |
| MNSSMRGDRGSL | 465 |
| MNSSMRGDRQAL | 466 |
| MNSSMRGDRQSL | 467 |
| MNSSMRGDRQYL | 468 |
| MNSSSRGDRAAL | 469 |
| MNSSTRGDRAAL | 470 |
| MNSSTRGDRASL | 471 |
| MNSSTRGDRQAL | 472 |
| MNSSTRGDRQSL | 473 |
| MNSSTRGDRQYL | 474 |
| MNSSVRGDRAAL | 475 |
| MNSSVRGDRAYL | 476 |
| MNSSVRGDRQAL | 477 |
| MNSSVRGDRQSL | 478 |
| MNSSVRGDRQYL | 479 |
| MNTSARGDRAYL | 480 |
| MNTSGRGDRAAL | 481 |
| MNTSGRGDRQAL | 482 |
| MNTSGRGDRQYL | 483 |
| MNTSMRGDRAYL | 484 |
| MNTSTRGDRQYL | 485 |
| MNYPHRGDRGAL | 486 |
| MNYSARGDRAAL | 487 |
| MNYSARGDRGYL | 488 |
| MNYSARGDRQAL | 489 |
| MNYSARGDRQSL | 490 |
| MNYSGRGDRAAL | 491 |
| MNYSGRGDRASL | 492 |
| MNYSGRGDRGSL | 493 |
| MNYSGRGDRQAL | 494 |
| MNYSGRGDRQSL | 495 |
| MNYSSRGDRQAL | 496 |
| MNYSTRGDRAAL | 497 |
| MNYSTRGDRQAL | 498 |
| MNYSTRGDRQSL | 499 |
| MNYSTRGDRQYL | 500 |
| NNSGMRGDRGAL | 501 |
| QNSGMRGDRGAL | 502 |
| SNASARGDRAAL | 503 |
| SNASARGDRASL | 504 |
| SNASARGDRAYL | 505 |
| SNASARGDRGAL | 506 |
| SNASARGDRGSL | 507 |
| SNASARGDRGYL | 508 |
| SNASARGDRQAL | 509 |
| SNASARGDRQSL | 510 |
| SNASARGDRQYL | 511 |
| SNASGRGDRAAL | 512 |
| SNASGRGDRASL | 513 |
| SNASGRGDRAYL | 514 |
| SNASGRGDRGAL | 515 |
| SNASGRGDRGSL | 516 |
| SNASGRGDRGYL | 517 |
| SNASGRGDRQAL | 518 |
| SNASGRGDRQSL | 519 |
| SNASGRGDRQYL | 520 |
| SNASIRGDRQLL | 521 |
| SNASMRGDRAAL | 522 |
| SNASMRGDRASL | 523 |
| SNASMRGDRGAL | 524 |
| SNASMRGDRGSL | 525 |
| SNASMRGDRQAL | 526 |
| SNASMRGDRQSL | 527 |
| SNASSRGDRAAL | 528 |
| SNASSRGDRASL | 529 |
| SNASSRGDRAYL | 530 |
| SNASSRGDRGAL | 531 |
| SNASSRGDRGSL | 532 |
| SNASSRGDRQAL | 533 |
| SNASSRGDRQSL | 534 |
| SNASSRGDRQYL | 535 |
| SNASTRGDRAAL | 536 |
| SNASTRGDRASL | 537 |
| SNASTRGDRAYL | 538 |
| SNASTRGDRGAL | 539 |
| SNASTRGDRGSL | 540 |
| SNASTRGDRGYL | 541 |
| SNASTRGDRQAL | 542 |
| SNASTRGDRQSL | 543 |
| SNASTRGDRQYL | 544 |
| SNASVRGDRAAL | 545 |
| SNASVRGDRASL | 546 |
| SNASVRGDRAYL | 547 |
| SNASVRGDRGSL | 548 |
| SNASVRGDRGYL | 549 |
| SNASVRGDRQAL | 550 |
| SNASVRGDRQSL | 551 |
| SNASVRGDRQYL | 552 |
| SNGNFRGDRQNL | 553 |
| SNGNVRGDRGML | 554 |
| SNGSARGDRGYL | 555 |
| SNGSARGDRQAL | 556 |
| SNGSARGDRQSL | 557 |
| SNGSARGDRQYL | 558 |
| SNGSGRGDRASL | 559 |
| SNGSGRGDRAYL | 560 |
| SNGSGRGDRGAL | 561 |
| SNGSGRGDRGSL | 562 |
| SNGSGRGDRGYL | 563 |
| SNGSGRGDRQAL | 564 |
| SNGSGRGDRQSL | 565 |
| SNGSGRGDRQYL | 566 |
| SNGSMRGDRAYL | 567 |
| SNGSMRGDRGYL | 568 |
| SNGSMRGDRQAL | 569 |
| SNGSMRGDRQSL | 570 |
| SNGSMRGDRQYL | 571 |
| SNGSSRGDRGYL | 572 |
| SNGSSRGDRQYL | 573 |
| SNGSTRGDRAAL | 574 |
| SNGSTRGDRASL | 575 |
| SNGSTRGDRGAL | 576 |
| SNGSTRGDRGYL | 577 |
| SNGSTRGDRQAL | 578 |
| SNGSTRGDRQSL | 579 |
| SNGSTRGDRQYL | 580 |
| SNGSVRGDRGYL | 581 |
| SNGSVRGDRQAL | 582 |
| SNGSVRGDRQSL | 583 |
| SNGSVRGDRQYL | 584 |
| SNSGMRGDRGAL | 585 |
| SNSSARGDRAYL | 586 |
| SNSSARGDRGAL | 587 |
| SNSSARGDRGIL | 588 |
| SNSSARGDRGSL | 589 |
| SNSSARGDRGYL | 590 |
| SNSSARGDRQAL | 591 |
| SNSSARGDRQSL | 592 |
| SNSSARGDRQYL | 593 |
| SNSSGRGDRAAL | 594 |
| SNSSGRGDRAYL | 595 |
| SNSSGRGDRGAL | 596 |
| SNSSGRGDRGSL | 597 |
| SNSSGRGDRGYL | 598 |
| SNSSGRGDRQAL | 599 |
| SNSSGRGDRQSL | 600 |
| SNSSGRGDRQYL | 601 |
| SNSSMRGDRAAL | 602 |
| SNSSMRGDRASL | 603 |
| SNSSMRGDRAYL | 604 |
| SNSSMRGDRGSL | 605 |
| SNSSMRGDRQAL | 606 |
| SNSSMRGDRQSL | 607 |
| SNSSMRGDRQYL | 608 |
| SNSSSRGDRAAL | 609 |
| SNSSSRGDRGSL | 610 |
| SNSSSRGDRQAL | 611 |
| SNSSSRGDRQSL | 612 |
| SNSSSRGDRQYL | 613 |
| SNSSTRGDRAAL | 614 |
| SNSSTRGDRASL | 615 |
| SNSSTRGDRGSL | 616 |
| SNSSTRGDRGYL | 617 |
| SNSSTRGDRQAL | 618 |
| SNSSTRGDRQSL | 619 |
| SNSSTRGDRQYL | 620 |
| SNSSVRGDRQAL | 621 |
| SNSSVRGDRQSL | 622 |
| SNSSVRGDRQYL | 623 |
| SNTSARGDRAAL | 624 |
| SNTSARGDRGAL | 625 |
| SNTSARGDRGSL | 626 |
| SNTSARGDRGYL | 627 |
| SNTSARGDRQAL | 628 |
| SNTSARGDRQSL | 629 |
| SNTSARGDRQYL | 630 |
| SNTSGRGDRAAL | 631 |
| SNTSGRGDRASL | 632 |
| SNTSGRGDRGAL | 633 |
| SNTSGRGDRGSL | 634 |
| SNTSGRGDRGYL | 635 |
| SNTSGRGDRQAL | 636 |
| SNTSGRGDRQSL | 637 |
| SNTSGRGDRQYL | 638 |
| SNTSMRGDRAYL | 639 |
| SNTSMRGDRGSL | 640 |
| SNTSMRGDRQAL | 641 |
| SNTSMRGDRQSL | 642 |
| SNTSSRGDRQAL | 643 |
| SNTSTRGDRQAL | 644 |
| SNTSTRGDRQSL | 645 |
| SNTSTRGDRQYL | 646 |
| SNTSVRGDRAYL | 647 |
| SNTSVRGDRQAL | 648 |
| SNTSVRGDRQSL | 649 |
| SNTSVRGDRQYL | 650 |
| SNYPHRGDRGAL | 651 |
| SNYSARGDRAAL | 652 |
| SNYSARGDRASL | 653 |
| SNYSARGDRGAL | 654 |
| SNYSARGDRGSL | 655 |
| SNYSARGDRQAL | 656 |
| SNYSARGDRQSL | 657 |
| SNYSARGDRQYL | 658 |
| SNYSGRGDRAAL | 659 |
| SNYSGRGDRGAL | 660 |
| SNYSGRGDRGSL | 661 |
| SNYSGRGDRQAL | 662 |
| SNYSGRGDRQSL | 663 |
| SNYSMRGDRAAL | 664 |
| SNYSMRGDRASL | 665 |
| SNYSMRGDRGSL | 666 |
| SNYSMRGDRQAL | 667 |
| SNYSMRGDRQSL | 668 |
| SNYSSRGDRGAL | 669 |
| SNYSSRGDRGSL | 670 |
| SNYSSRGDRQAL | 671 |
| SNYSSRGDRQSL | 672 |
| SNYSTRGDRAAL | 673 |
| SNYSTRGDRGAL | 674 |
| SNYSTRGDRGSL | 675 |
| SNYSTRGDRQAL | 676 |
| SNYSTRGDRQSL | 677 |
| SNYSTRGDRQYL | 678 |
| SNYSVRGDRQAL | 679 |
| SNYSVRGDRQSL | 680 |
| SNYSVRGDRQYL | 681 |
| TNSGMRGDRGAL | 682 |
| VNASARGDRAAL | 683 |
| VNASARGDRGAL | 684 |
| VNASARGDRGSL | 685 |
| VNASARGDRGYL | 686 |
| VNASARGDRQAL | 687 |
| VNASARGDRQYL | 688 |
| VNASGRGDRAAL | 689 |
| VNASGRGDRAYL | 690 |
| VNASGRGDRGAL | 691 |
| VNASGRGDRGSL | 692 |
| VNASGRGDRGYL | 693 |
| VNASGRGDRQAL | 694 |
| VNASGRGDRQYL | 695 |
| VNASMRGDRAYL | 696 |
| VNASMRGDRQAL | 697 |
| VNASMRGDRQSL | 698 |
| VNASSRGDRGSL | 699 |
| VNASSRGDRQAL | 700 |
| VNASSRGDRQYL | 701 |
| VNASTRGDRAAL | 702 |
| VNASTRGDRGAL | 703 |
| VNASTRGDRGSL | 704 |
| VNASTRGDRGYL | 705 |
| VNASTRGDRQAL | 706 |
| VNASTRGDRQSL | 707 |
| VNASTRGDRQYL | 708 |
| VNASVRGDRGSL | 709 |
| VNASVRGDRGYL | 710 |
| VNASVRGDRQAL | 711 |
| VNASVRGDRQSL | 712 |
| VNASVRGDRQYL | 713 |
| VNGNFRGDRQNL | 714 |
| VNGNVRGDRGML | 715 |
| VNGSARGDRAYL | 716 |
| VNGSARGDRGAL | 717 |
| VNGSARGDRGSL | 718 |
| VNGSARGDRGYL | 719 |
| VNGSARGDRQAL | 720 |
| VNGSARGDRQSL | 721 |
| VNGSARGDRQYL | 722 |
| VNGSGRGDRAAL | 723 |
| VNGSGRGDRASL | 724 |
| VNGSGRGDRAYL | 725 |
| VNGSGRGDRGAL | 726 |
| VNGSGRGDRGSL | 727 |
| VNGSGRGDRGYL | 728 |
| VNGSGRGDRQAL | 729 |
| VNGSGRGDRQSL | 730 |
| VNGSGRGDRQYL | 731 |
| VNGSMRGDRGYL | 732 |
| VNGSMRGDRQAL | 733 |
| VNGSMRGDRQSL | 734 |
| VNGSMRGDRQYL | 735 |
| VNGSSRGDRAYL | 736 |
| VNGSSRGDRGAL | 737 |
| VNGSSRGDRGSL | 738 |
| VNGSSRGDRQAL | 739 |
| VNGSSRGDRQSL | 740 |
| VNGSSRGDRQYL | 741 |
| VNGSTRGDRAAL | 742 |
| VNGSTRGDRAYL | 743 |
| VNGSTRGDRGAL | 744 |
| VNGSTRGDRGYL | 745 |
| VNGSTRGDRQAL | 746 |
| VNGSTRGDRQSL | 747 |
| VNGSTRGDRQYL | 748 |
| VNGSVRGDRAYL | 749 |
| VNGSVRGDRQAL | 750 |
| VNGSVRGDRQSL | 751 |
| VNGSVRGDRQYL | 752 |
| VNSGMRGDRGAL | 753 |
| VNSSARGDRGAL | 754 |
| VNSSARGDRGSL | 755 |
| VNSSARGDRGYL | 756 |
| VNSSARGDRQAL | 757 |
| VNSSARGDRQSL | 758 |
| VNSSARGDRQYL | 759 |
| VNSSGRGDRAAL | 760 |
| VNSSGRGDRAYL | 761 |
| VNSSGRGDRGAL | 762 |
| VNSSGRGDRQAL | 763 |
| VNSSGRGDRQSL | 764 |
| VNSSGRGDRQYL | 765 |
| VNSSMRGDRAYL | 766 |
| VNSSMRGDRQAL | 767 |
| VNSSMRGDRQSL | 768 |
| VNSSMRGDRQYL | 769 |
| VNSSSRGDRGSL | 770 |
| VNSSSRGDRQAL | 771 |
| VNSSSRGDRQSL | 772 |
| VNSSTRGDRGAL | 773 |
| VNSSTRGDRQAL | 774 |
| VNSSTRGDRQSL | 775 |
| VNSSTRGDRQYL | 776 |
| VNSSVRGDRAYL | 777 |
| VNSSVRGDRQAL | 778 |
| VNSSVRGDRQSL | 779 |
| VNTSARGDRAYL | 780 |
| VNTSGRGDRAAL | 781 |
| VNTSGRGDRGAL | 782 |
| VNTSGRGDRGSL | 783 |
| VNTSGRGDRGYL | 784 |
| VNTSGRGDRQAL | 785 |
| VNTSGRGDRQSL | 786 |
| VNTSGRGDRQYL | 787 |
| VNTSTRGDRAYL | 788 |
| VNYPHRGDRGAL | 789 |
| VNYSARGDRGAL | 790 |
| VNYSARGDRGSL | 791 |
| VNYSARGDRQAL | 792 |
| VNYSARGDRQSL | 793 |
| VNYSGRGDRAAL | 794 |
| VNYSGRGDRGAL | 795 |
| VNYSGRGDRQAL | 796 |
| VNYSGRGDRQSL | 797 |
| VNYSGRGDRQYL | 798 |
| VNYSSRGDRGSL | 799 |
| VNYSSRGDRGYL | 800 |

TABLE 1b
Split Amino Acid Sequences from Table 1a

| 5 aa | SEQ | | SEQ |
|---|---|---|---|
| substi- | ID | 7 aa | ID |
| tution | NO | insert | NO |
| ANASA | 801 | RGDRAAL | 1600 |
| ANASA | 802 | RGDRGSL | 1601 |
| ANASA | 803 | RGDRGYL | 1602 |
| ANASA | 804 | RGDRQAL | 1603 |
| ANASA | 805 | RGDRQSL | 1604 |
| ANASA | 806 | RGDRQYL | 1605 |
| ANASG | 807 | RGDRAAL | 1606 |
| ANASG | 808 | RGDRASL | 1607 |
| ANASG | 809 | RGDRAYL | 1608 |
| ANASG | 810 | RGDRGAL | 1609 |
| ANASG | 811 | RGDRGSL | 1610 |
| ANASG | 812 | RGDRGYL | 1611 |
| ANASG | 813 | RGDRQAL | 1612 |
| ANASG | 814 | RGDRQSL | 1613 |
| ANASG | 815 | RGDRQYL | 1614 |
| ANASM | 816 | RGDRAAL | 1615 |
| ANASM | 817 | RGDRASL | 1616 |
| ANASM | 818 | RGDRAYL | 1617 |
| ANASM | 819 | RGDRQAL | 1618 |
| ANASM | 820 | RGDRQSL | 1619 |
| ANASS | 821 | RGDRGSL | 1620 |
| ANASS | 822 | RGDRGYL | 1621 |
| ANASS | 823 | RGDRQAL | 1622 |
| ANASS | 824 | RGDRQSL | 1623 |
| ANASS | 825 | RGDRQYL | 1624 |
| ANAST | 826 | RGDRASL | 1625 |
| ANAST | 827 | RGDRAYL | 1626 |
| ANAST | 828 | RGDRGAL | 1627 |
| ANAST | 829 | RGDRGSL | 1628 |
| ANAST | 830 | RGDRGYL | 1629 |
| ANAST | 831 | RGDRQAL | 1630 |
| ANAST | 832 | RGDRQSL | 1631 |
| ANAST | 833 | RGDRQYL | 1632 |
| ANASV | 834 | RGDRASL | 1633 |
| ANASV | 835 | RGDRGYL | 1634 |
| ANASV | 836 | RGDRQAL | 1635 |
| ANASV | 837 | RGDRQSL | 1636 |
| ANASV | 838 | RGDRQYL | 1637 |
| ANGNF | 839 | RGDRQNL | 1638 |
| ANGNV | 840 | RGDRGML | 1639 |
| ANGSA | 841 | RGDRAYL | 1640 |
| ANGSA | 842 | RGDRGSL | 1641 |
| ANGSA | 843 | RGDRGYL | 1642 |
| ANGSA | 844 | RGDRQAL | 1643 |
| ANGSA | 845 | RGDRQYL | 1644 |
| ANGSG | 846 | RGDRAAL | 1645 |
| ANGSG | 847 | RGDRAYL | 1646 |
| ANGSG | 848 | RGDRGAL | 1647 |
| ANGSG | 849 | RGDRGSL | 1648 |
| ANGSG | 850 | RGDRGYL | 1649 |
| ANGSG | 851 | RGDRQAL | 1650 |
| ANGSG | 852 | RGDRQSL | 1651 |
| ANGSG | 853 | RGDRQYL | 1652 |
| ANGSM | 854 | RGDRGYL | 1653 |
| ANGSM | 855 | RGDRQAL | 1654 |
| ANGSM | 856 | RGDRQSL | 1655 |
| ANGSM | 857 | RGDRQYL | 1656 |
| ANGSS | 858 | RGDRAYL | 1657 |
| ANGSS | 859 | RGDRGSL | 1658 |
| ANGSS | 860 | RGDRGYL | 1659 |
| ANGSS | 861 | RGDRQAL | 1660 |
| ANGSS | 862 | RGDRQSL | 1661 |
| ANGSS | 863 | RGDRQYL | 1662 |
| ANGST | 864 | RGDRAAL | 1663 |
| ANGST | 865 | RGDRAYL | 1664 |
| ANGST | 866 | RGDRGAL | 1665 |
| ANGST | 867 | RGDRGYL | 1666 |
| ANGST | 868 | RGDRQAL | 1667 |
| ANGST | 869 | RGDRQSL | 1668 |
| ANGST | 870 | RGDRQYL | 1669 |
| ANGSV | 871 | RGDRAYL | 1670 |
| ANGSV | 872 | RGDRGYL | 1671 |
| ANGSV | 873 | RGDRQAL | 1672 |
| ANGSV | 874 | RGDRQYL | 1673 |
| ANSGM | 875 | RGDRGAL | 1674 |
| ANSSA | 876 | RGDRGAL | 1675 |
| ANSSA | 877 | RGDRGSL | 1676 |
| ANSSA | 878 | RGDRGYL | 1677 |
| ANSSA | 879 | RGDRQAL | 1678 |
| ANSSA | 880 | RGDRQSL | 1679 |
| ANSSA | 881 | RGDRQYL | 1680 |
| ANSSG | 882 | RGDRAAL | 1681 |
| ANSSG | 883 | RGDRASL | 1682 |
| ANSSG | 884 | RGDRAYL | 1683 |
| ANSSG | 885 | RGDRGAL | 1684 |
| ANSSG | 886 | RGDRGSL | 1685 |
| ANSSG | 887 | RGDRGYL | 1686 |
| ANSSG | 888 | RGDRQAL | 1687 |
| ANSSG | 889 | RGDRQSL | 1688 |
| ANSSG | 890 | RGDRQYL | 1689 |
| ANSSM | 891 | RGDRAYL | 1690 |
| ANSSM | 892 | RGDRQAL | 1691 |
| ANSSM | 893 | RGDRQSL | 1692 |
| ANSSM | 894 | RGDRQYL | 1693 |
| ANSSS | 895 | RGDRGSL | 1694 |
| ANSSS | 896 | RGDRGYL | 1695 |
| ANSSS | 897 | RGDRQAL | 1696 |
| ANSSS | 898 | RGDRQSL | 1697 |
| ANSSS | 899 | RGDRQYL | 1698 |
| ANSST | 900 | RGDRAAL | 1699 |
| ANSST | 901 | RGDRGSL | 1700 |
| ANSST | 902 | RGDRQAL | 1701 |
| ANSST | 903 | RGDRQSL | 1702 |
| ANSST | 904 | RGDRQYL | 1703 |
| ANSSV | 905 | RGDRAYL | 1704 |
| ANSSV | 906 | RGDRGYL | 1705 |
| ANSSV | 907 | RGDRQAL | 1706 |
| ANSSV | 908 | RGDRQSL | 1707 |
| ANSSV | 909 | RGDRQYL | 1708 |
| ANTSA | 910 | RGDRAYL | 1709 |
| ANTSA | 911 | RGDRGAL | 1710 |
| ANTSA | 912 | RGDRGSL | 1711 |
| ANTSA | 913 | RGDRGYL | 1712 |
| ANTSA | 914 | RGDRQAL | 1713 |
| ANTSA | 915 | RGDRQYL | 1714 |
| ANTSG | 916 | RGDRASL | 1715 |
| ANTSG | 917 | RGDRGAL | 1716 |
| ANTSG | 918 | RGDRGSL | 1717 |
| ANTSG | 919 | RGDRGYL | 1718 |
| ANTSG | 920 | RGDRQAL | 1719 |
| ANTSG | 921 | RGDRQSL | 1720 |
| ANTSG | 922 | RGDRQYL | 1721 |
| ANTSM | 923 | RGDRAYL | 1722 |
| ANTSM | 924 | RGDRQAL | 1723 |
| ANTSM | 925 | RGDRQSL | 1724 |
| ANTSM | 926 | RGDRQYL | 1725 |
| ANTSS | 927 | RGDRGSL | 1726 |
| ANTSS | 928 | RGDRQAL | 1727 |
| ANTSS | 929 | RGDRQYL | 1728 |
| ANTST | 930 | RGDRAAL | 1729 |
| ANTST | 931 | RGDRQAL | 1730 |
| ANTST | 932 | RGDRQSL | 1731 |
| ANTST | 933 | RGDRQYL | 1732 |
| ANTSV | 934 | RGDRAYL | 1733 |
| ANTSV | 935 | RGDRGSL | 1734 |
| ANTSV | 936 | RGDRQAL | 1735 |
| ANTSV | 937 | RGDRQSL | 1736 |
| ANTSV | 938 | RGDRQYL | 1737 |
| ANYPH | 939 | RGDRGAL | 1738 |
| ANYSA | 940 | RGDRAAL | 1739 |
| ANYSA | 941 | RGDRASL | 1740 |
| ANYSA | 942 | RGDRGAL | 1741 |
| ANYSA | 943 | RGDRGSL | 1742 |
| ANYSA | 944 | RGDRQAL | 1743 |
| ANYSA | 945 | RGDRQSL | 1744 |
| ANYSG | 946 | RGDRAAL | 1745 |
| ANYSG | 947 | RGDRASL | 1746 |
| ANYSG | 948 | RGDRGAL | 1747 |
| ANYSG | 949 | RGDRGSL | 1748 |
| ANYSG | 950 | RGDRQAL | 1749 |
| ANYSG | 951 | RGDRQSL | 1750 |
| ANYSG | 952 | RGDRQYL | 1751 |
| ANYSM | 953 | RGDRAAL | 1752 |
| ANYSM | 954 | RGDRASL | 1753 |
| ANYSM | 955 | RGDRAYL | 1754 |
| ANYSM | 956 | RGDRQAL | 1755 |
| ANYSM | 957 | RGDRQSL | 1756 |
| ANYSS | 958 | RGDRGAL | 1757 |
| ANYSS | 959 | RGDRGSL | 1758 |
| ANYSS | 960 | RGDRQAL | 1759 |
| ANYSS | 961 | RGDRQSL | 1760 |
| ANYSS | 962 | RGDRQYL | 1761 |
| ANYST | 963 | RGDRQAL | 1762 |
| ANYST | 964 | RGDRQSL | 1763 |
| ANYST | 965 | RGDRQYL | 1764 |
| ANYSV | 966 | RGDRQAL | 1765 |
| ANYSV | 967 | RGDRQSL | 1766 |
| ANYSV | 968 | RGDRQYL | 1767 |
| FNSGM | 969 | RGDRGAL | 1768 |
| GNSGM | 970 | RGDRGAL | 1769 |
| HNSGM | 971 | RGDRGAL | 1770 |
| LNASA | 972 | RGDRAAL | 1771 |
| LNASA | 973 | RGDRASL | 1772 |
| LNASA | 974 | RGDRAYL | 1773 |
| LNASA | 975 | RGDRGAL | 1774 |
| LNASA | 976 | RGDRGSL | 1775 |
| LNASA | 977 | RGDRGYL | 1776 |
| LNASA | 978 | RGDRQAL | 1777 |
| LNASA | 979 | RGDRQSL | 1778 |
| LNASA | 980 | RGDRQYL | 1779 |
| LNASG | 981 | RGDRAAL | 1780 |
| LNASG | 982 | RGDRASL | 1781 |
| LNASG | 983 | RGDRAYL | 1782 |
| LNASG | 984 | RGDRGAL | 1783 |
| LNASG | 985 | RGDRGSL | 1784 |
| LNASG | 986 | RGDRGYL | 1785 |
| LNASG | 987 | RGDRQAL | 1786 |
| LNASG | 988 | RGDRQSL | 1787 |
| LNASG | 989 | RGDRQYL | 1788 |
| LNASI | 990 | RGDRQLL | 1789 |
| LNASM | 991 | RGDRAAL | 1790 |
| LNASM | 992 | RGDRASL | 1791 |
| LNASM | 993 | RGDRAYL | 1792 |
| LNASM | 994 | RGDRGAL | 1793 |
| LNASM | 995 | RGDRGSL | 1794 |
| LNASM | 996 | RGDRGYL | 1795 |
| LNASM | 997 | RGDRQAL | 1796 |
| LNASM | 998 | RGDRQSL | 1797 |
| LNASM | 999 | RGDRQYL | 1798 |
| LNASS | 1000 | RGDRAAL | 1799 |
| LNASS | 1001 | RGDRASL | 1800 |
| LNASS | 1002 | RGDRGAL | 1801 |
| LNASS | 1003 | RGDRGSL | 1802 |
| LNASS | 1004 | RGDRQAL | 1803 |
| LNASS | 1005 | RGDRQSL | 1804 |
| LNASS | 1006 | RGDRQYL | 1805 |
| LNAST | 1007 | RGDRAAL | 1806 |
| LNAST | 1008 | RGDRASL | 1807 |
| LNAST | 1009 | RGDRGAL | 1808 |
| LNAST | 1010 | RGDRGSL | 1809 |
| LNAST | 1011 | RGDRGYL | 1810 |
| LNAST | 1012 | RGDRQAL | 1811 |
| LNAST | 1013 | RGDRQSL | 1812 |
| LNAST | 1014 | RGDRQYL | 1813 |
| LNASV | 1015 | RGDRAAL | 1814 |
| LNASV | 1016 | RGDRASL | 1815 |
| LNASV | 1017 | RGDRGAL | 1816 |
| LNASV | 1018 | RGDRGSL | 1817 |
| LNASV | 1019 | RGDRQAL | 1818 |
| LNASV | 1020 | RGDRQSL | 1819 |
| LNASV | 1021 | RGDRQYL | 1820 |
| LNGNF | 1022 | RGDRQNL | 1821 |
| LNGNV | 1023 | RGDRGML | 1822 |
| LNGSA | 1024 | RGDRAAL | 1823 |
| LNGSA | 1025 | RGDRAYL | 1824 |
| LNGSA | 1026 | RGDRGSL | 1825 |
| LNGSA | 1027 | RGDRGYL | 1826 |
| LNGSA | 1028 | RGDRQAL | 1827 |
| LNGSA | 1029 | RGDRQSL | 1828 |
| LNGSA | 1030 | RGDRQYL | 1829 |
| LNGSG | 1031 | RGDRAAL | 1830 |
| LNGSG | 1032 | RGDRAYL | 1831 |
| LNGSG | 1033 | RGDRGAL | 1832 |
| LNGSG | 1034 | RGDRGSL | 1833 |
| LNGSG | 1035 | RGDRGYL | 1834 |
| LNGSG | 1036 | RGDRQAL | 1835 |
| LNGSG | 1037 | RGDRQYL | 1836 |
| LNGSM | 1038 | RGDRASL | 1837 |
| LNGSM | 1039 | RGDRGYL | 1838 |
| LNGSM | 1040 | RGDRQAL | 1839 |
| LNGSM | 1041 | RGDRQSL | 1840 |
| LNGSM | 1042 | RGDRQYL | 1841 |
| LNGSS | 1043 | RGDRAAL | 1842 |
| LNGSS | 1044 | RGDRAYL | 1843 |
| LNGSS | 1045 | RGDRGAL | 1844 |
| LNGSS | 1046 | RGDRQYL | 1845 |
| LNGST | 1047 | RGDRAAL | 1846 |
| LNGST | 1048 | RGDRASL | 1847 |
| LNGST | 1049 | RGDRAYL | 1848 |
| LNGST | 1050 | RGDRGAL | 1849 |
| LNGST | 1051 | RGDRGSL | 1850 |
| LNGST | 1052 | RGDRGYL | 1851 |
| LNGST | 1053 | RGDRQAL | 1852 |
| LNGST | 1054 | RGDRQSL | 1853 |
| LNGST | 1055 | RGDRQYL | 1854 |
| LNGSV | 1056 | RGDRASL | 1855 |
| LNGSV | 1057 | RGDRAYL | 1856 |
| LNGSV | 1058 | RGDRGYL | 1857 |
| LNGSV | 1059 | RGDRQAL | 1858 |
| LNGSV | 1060 | RGDRQSL | 1859 |
| LNGSV | 1061 | RGDRQYL | 1860 |
| LNSGM | 1062 | RGDRGAL | 1861 |
| LNSSA | 1063 | RGDRASL | 1862 |
| LNSSA | 1064 | RGDRAYL | 1863 |
| LNSSA | 1065 | RGDRGAL | 1864 |
| LNSSA | 1066 | RGDRGIL | 1865 |
| LNSSA | 1067 | RGDRGSL | 1866 |
| LNSSA | 1068 | RGDRGYL | 1867 |
| LNSSA | 1069 | RGDRQAL | 1868 |
| LNSSA | 1070 | RGDRQSL | 1869 |
| LNSSA | 1071 | RGDRQYL | 1870 |
| LNSSG | 1072 | RGDRAAL | 1871 |
| LNSSG | 1073 | RGDRASL | 1872 |
| LNSSG | 1074 | RGDRAYL | 1873 |
| LNSSG | 1075 | RGDRGAL | 1874 |
| LNSSG | 1076 | RGDRGSL | 1875 |
| LNSSG | 1077 | RGDRGYL | 1876 |
| LNSSG | 1078 | RGDRQAL | 1877 |
| LNSSG | 1079 | RGDRQSL | 1878 |
| LNSSG | 1080 | RGDRQYL | 1879 |
| LNSSM | 1081 | RGDRAAL | 1880 |
| LNSSM | 1082 | RGDRASL | 1881 |
| LNSSM | 1083 | RGDRAYL | 1882 |
| LNSSM | 1084 | RGDRGAL | 1883 |
| LNSSM | 1085 | RGDRGSL | 1884 |
| LNSSM | 1086 | RGDRQAL | 1885 |
| LNSSM | 1087 | RGDRQSL | 1886 |
| LNSSM | 1088 | RGDRQYL | 1887 |
| LNSSS | 1089 | RGDRAAL | 1888 |
| LNSSS | 1090 | RGDRGAL | 1889 |
| LNSSS | 1091 | RGDRGSL | 1890 |
| LNSSS | 1092 | RGDRQAL | 1891 |
| LNSSS | 1093 | RGDRQSL | 1892 |
| LNSSS | 1094 | RGDRQYL | 1893 |
| LNSST | 1095 | RGDRAAL | 1894 |
| LNSST | 1096 | RGDRASL | 1895 |
| LNSST | 1097 | RGDRGAL | 1896 |
| LNSST | 1098 | RGDRGSL | 1897 |
| LNSST | 1099 | RGDRQAL | 1898 |
| LNSST | 1100 | RGDRQSL | 1899 |
| LNSST | 1101 | RGDRQYL | 1900 |
| LNSSV | 1102 | RGDRAAL | 1901 |
| LNSSV | 1103 | RGDRASL | 1902 |
| LNSSV | 1104 | RGDRGAL | 1903 |
| LNSSV | 1105 | RGDRGSL | 1904 |
| LNSSV | 1106 | RGDRGYL | 1905 |
| LNSSV | 1107 | RGDRQAL | 1906 |
| LNSSV | 1108 | RGDRQSL | 1907 |
| LNSSV | 1109 | RGDRQYL | 1908 |
| LNTSA | 1110 | RGDRAAL | 1909 |
| LNTSA | 1111 | RGDRASL | 1910 |
| LNTSA | 1112 | RGDRAYL | 1911 |
| LNTSA | 1113 | RGDRGAL | 1912 |
| LNTSA | 1114 | RGDRGSL | 1913 |
| LNTSA | 1115 | RGDRGYL | 1914 |
| LNTSA | 1116 | RGDRQAL | 1915 |
| LNTSA | 1117 | RGDRQSL | 1916 |
| LNTSA | 1118 | RGDRQYL | 1917 |
| LNTSG | 1119 | RGDRAAL | 1918 |
| LNTSG | 1120 | RGDRASL | 1919 |
| LNTSG | 1121 | RGDRAYL | 1920 |
| LNTSG | 1122 | RGDRGAL | 1921 |
| LNTSG | 1123 | RGDRGSL | 1922 |
| LNTSG | 1124 | RGDRGYL | 1923 |
| LNTSG | 1125 | RGDRQAL | 1924 |
| LNTSG | 1126 | RGDRQSL | 1925 |
| LNTSG | 1127 | RGDRQYL | 1926 |
| LNTSM | 1128 | RGDRAAL | 1927 |
| LNTSM | 1129 | RGDRASL | 1928 |
| LNTSM | 1130 | RGDRAYL | 1929 |
| LNTSM | 1131 | RGDRGAL | 1930 |
| LNTSM | 1132 | RGDRGSL | 1931 |
| LNTSM | 1133 | RGDRQAL | 1932 |
| LNTSM | 1134 | RGDRQSL | 1933 |
| LNTSM | 1135 | RGDRQYL | 1934 |
| LNTSS | 1136 | RGDRAAL | 1935 |
| LNTSS | 1137 | RGDRGSL | 1936 |
| LNTSS | 1138 | RGDRQAL | 1937 |
| LNTSS | 1139 | RGDRQSL | 1938 |
| LNTSS | 1140 | RGDRQYL | 1939 |
| LNTST | 1141 | RGDRAAL | 1940 |
| LNTST | 1142 | RGDRASL | 1941 |
| LNTST | 1143 | RGDRGAL | 1942 |
| LNTST | 1144 | RGDRGSL | 1943 |
| LNTST | 1145 | RGDRQAL | 1944 |
| LNTST | 1146 | RGDRQSL | 1945 |
| LNTST | 1147 | RGDRQYL | 1946 |
| LNTSV | 1148 | RGDRAAL | 1947 |
| LNTSV | 1149 | RGDRASL | 1948 |
| LNTSV | 1150 | RGDRAYL | 1949 |
| LNTSV | 1151 | RGDRGAL | 1950 |
| LNTSV | 1152 | RGDRGSL | 1951 |
| LNTSV | 1153 | RGDRQAL | 1952 |
| LNTSV | 1154 | RGDRQSL | 1953 |
| LNYPH | 1155 | RGDRGAL | 1954 |
| LNYSA | 1156 | RGDRGAL | 1955 |
| LNYSA | 1157 | RGDRGSL | 1956 |
| LNYSA | 1158 | RGDRQAL | 1957 |
| LNYSA | 1159 | RGDRQSL | 1958 |
| LNYSA | 1160 | RGDRQYL | 1959 |
| LNYSG | 1161 | RGDRAAL | 1960 |
| LNYSG | 1162 | RGDRASL | 1961 |
| LNYSG | 1163 | RGDRGAL | 1962 |
| LNYSG | 1164 | RGDRGSL | 1963 |
| LNYSG | 1165 | RGDRQAL | 1964 |
| LNYSG | 1166 | RGDRQSL | 1965 |
| LNYSG | 1167 | RGDRQYL | 1966 |
| LNYSM | 1168 | RGDRQAL | 1967 |
| LNYSM | 1169 | RGDRQSL | 1968 |
| LNYSS | 1170 | RGDRGAL | 1969 |
| LNYSS | 1171 | RGDRGSL | 1970 |
| LNYSS | 1172 | RGDRQAL | 1971 |
| LNYSS | 1173 | RGDRQSL | 1972 |
| LNYSS | 1174 | RGDRQYL | 1973 |
| LNYST | 1175 | RGDRASL | 1974 |
| LNYST | 1176 | RGDRGAL | 1975 |
| LNYST | 1177 | RGDRGSL | 1976 |
| LNYST | 1178 | RGDRQAL | 1977 |
| LNYST | 1179 | RGDRQSL | 1978 |
| LNYST | 1180 | RGDRQYL | 1979 |
| LNYSV | 1181 | RGDRQAL | 1980 |
| LNYSV | 1182 | RGDRQSL | 1981 |
| MNASA | 1183 | RGDRAAL | 1982 |
| MNASA | 1184 | RGDRAYL | 1983 |
| MNASA | 1185 | RGDRGSL | 1984 |
| MNASA | 1186 | RGDRGYL | 1985 |
| MNASA | 1187 | RGDRQAL | 1986 |
| MNASA | 1188 | RGDRQSL | 1987 |
| MNASA | 1189 | RGDRQYL | 1988 |
| MNASG | 1190 | RGDRAAL | 1989 |
| MNASG | 1191 | RGDRASL | 1990 |
| MNASG | 1192 | RGDRAYL | 1991 |
| MNASG | 1193 | RGDRGSL | 1992 |
| MNASG | 1194 | RGDRQAL | 1993 |
| MNASG | 1195 | RGDRQSL | 1994 |
| MNASG | 1196 | RGDRQYL | 1995 |
| MNASI | 1197 | RGDRQLL | 1996 |
| MNASM | 1198 | RGDRAAL | 1997 |
| MNASM | 1199 | RGDRAYL | 1998 |
| MNASM | 1200 | RGDRGAL | 1999 |
| MNASM | 1201 | RGDRQAL | 2000 |
| MNASM | 1202 | RGDRQSL | 2001 |
| MNASS | 1203 | RGDRAAL | 2002 |
| MNAST | 1204 | RGDRAAL | 2003 |
| MNAST | 1205 | RGDRASL | 2004 |
| MNAST | 1206 | RGDRAYL | 2005 |
| MNAST | 1207 | RGDRGAL | 2006 |
| MNAST | 1208 | RGDRGSL | 2007 |
| MNAST | 1209 | RGDRQAL | 2008 |
| MNAST | 1210 | RGDRQSL | 2009 |
| MNAST | 1211 | RGDRQYL | 2010 |
| MNASV | 1212 | RGDRAAL | 2011 |
| MNASV | 1213 | RGDRASL | 2012 |
| MNASV | 1214 | RGDRAYL | 2013 |
| MNASV | 1215 | RGDRQAL | 2014 |
| MNASV | 1216 | RGDRQSL | 2015 |
| MNASV | 1217 | RGDRQYL | 2016 |
| MNGNF | 1218 | RGDRQNL | 2017 |
| MNGSA | 1219 | RGDRAYL | 2018 |
| MNGSA | 1220 | RGDRGYL | 2019 |
| MNGSA | 1221 | RGDRQAL | 2020 |
| MNGSA | 1222 | RGDRQSL | 2021 |
| MNGSA | 1223 | RGDRQYL | 2022 |
| MNGSG | 1224 | RGDRAAL | 2023 |
| MNGSG | 1225 | RGDRAYL | 2024 |
| MNGSG | 1226 | RGDRGAL | 2025 |
| MNGSG | 1227 | RGDRGSL | 2026 |
| MNGSG | 1228 | RGDRGYL | 2027 |
| MNGSG | 1229 | RGDRQAL | 2028 |
| MNGSG | 1230 | RGDRQSL | 2029 |
| MNGSG | 1231 | RGDRQYL | 2030 |
| MNGSM | 1232 | RGDRQAL | 2031 |
| MNGSM | 1233 | RGDRQSL | 2032 |
| MNGSM | 1234 | RGDRQYL | 2033 |
| MNGSS | 1235 | RGDRQYL | 2034 |
| MNGST | 1236 | RGDRAYL | 2035 |
| MNGST | 1237 | RGDRGAL | 2036 |
| MNGST | 1238 | RGDRGYL | 2037 |
| MNGST | 1239 | RGDRQAL | 2038 |
| MNGST | 1240 | RGDRQSL | 2039 |
| MNGST | 1241 | RGDRQYL | 2040 |
| MNGSV | 1242 | RGDRGYL | 2041 |
| MNGSV | 1243 | RGDRQAL | 2042 |
| MNGSV | 1244 | RGDRQYL | 2043 |
| MNSGM | 1245 | RGDRGAL | 2044 |
| MNSSA | 1246 | RGDRAAL | 2045 |
| MNSSA | 1247 | RGDRAYL | 2046 |
| MNSSA | 1248 | RGDRGSL | 2047 |
| MNSSA | 1249 | RGDRGYL | 2048 |
| MNSSA | 1250 | RGDRQAL | 2049 |
| MNSSA | 1251 | RGDRQSL | 2050 |
| MNSSA | 1252 | RGDRQYL | 2051 |
| MNSSG | 1253 | RGDRAAL | 2052 |
| MNSSG | 1254 | RGDRASL | 2053 |
| MNSSG | 1255 | RGDRAYL | 2054 |
| MNSSG | 1256 | RGDRGAL | 2055 |
| MNSSG | 1257 | RGDRGSL | 2056 |
| MNSSG | 1258 | RGDRQAL | 2057 |
| MNSSG | 1259 | RGDRQSL | 2058 |
| MNSSG | 1260 | RGDRQYL | 2059 |
| MNSSM | 1261 | RGDRAAL | 2060 |
| MNSSM | 1262 | RGDRASL | 2061 |
| MNSSM | 1263 | RGDRAYL | 2062 |
| MNSSM | 1264 | RGDRGSL | 2063 |
| MNSSM | 1265 | RGDRQAL | 2064 |
| MNSSM | 1266 | RGDRQSL | 2065 |
| MNSSM | 1267 | RGDRQYL | 2066 |
| MNSSS | 1268 | RGDRAAL | 2067 |
| MNSST | 1269 | RGDRAAL | 2068 |
| MNSST | 1270 | RGDRASL | 2069 |
| MNSST | 1271 | RGDRQAL | 2070 |
| MNSST | 1272 | RGDRQSL | 2071 |
| MNSST | 1273 | RGDRQYL | 2072 |
| MNSSV | 1274 | RGDRAAL | 2073 |
| MNSSV | 1275 | RGDRAYL | 2074 |
| MNSSV | 1276 | RGDRQAL | 2075 |
| MNSSV | 1277 | RGDRQSL | 2076 |
| MNSSV | 1278 | RGDRQYL | 2077 |
| MNTSA | 1279 | RGDRAYL | 2078 |
| MNTSG | 1280 | RGDRAAL | 2079 |
| MNTSG | 1281 | RGDRQAL | 2080 |
| MNTSG | 1282 | RGDRQYL | 2081 |
| MNTSM | 1283 | RGDRAYL | 2082 |
| MNTST | 1284 | RGDRQYL | 2083 |
| MNYPH | 1285 | RGDRGAL | 2084 |
| MNYSA | 1286 | RGDRAAL | 2085 |
| MNYSA | 1287 | RGDRGYL | 2086 |
| MNYSA | 1288 | RGDRQAL | 2087 |
| MNYSA | 1289 | RGDRQSL | 2088 |
| MNYSG | 1290 | RGDRAAL | 2089 |
| MNYSG | 1291 | RGDRASL | 2090 |
| MNYSG | 1292 | RGDRGSL | 2091 |
| MNYSG | 1293 | RGDRQAL | 2092 |
| MNYSG | 1294 | RGDRQSL | 2093 |
| MNYSS | 1295 | RGDRQAL | 2094 |
| MNYST | 1296 | RGDRAAL | 2095 |
| MNYST | 1297 | RGDRQAL | 2096 |
| MNYST | 1298 | RGDRQSL | 2097 |
| MNYST | 1299 | RGDRQYL | 2098 |
| NNSGM | 1300 | RGDRGAL | 2099 |
| QNSGM | 1301 | RGDRGAL | 2100 |
| SNASA | 1302 | RGDRAAL | 2101 |
| SNASA | 1303 | RGDRASL | 2102 |
| SNASA | 1304 | RGDRAYL | 2103 |
| SNASA | 1305 | RGDRGAL | 2104 |
| SNASA | 1306 | RGDRGSL | 2105 |
| SNASA | 1307 | RGDRGYL | 2106 |
| SNASA | 1308 | RGDRQAL | 2107 |
| SNASA | 1309 | RGDRQSL | 2108 |
| SNASA | 1310 | RGDRQYL | 2109 |
| SNASG | 1311 | RGDRAAL | 2110 |
| SNASG | 1312 | RGDRASL | 2111 |
| SNASG | 1313 | RGDRAYL | 2112 |
| SNASG | 1314 | RGDRGAL | 2113 |
| SNASG | 1315 | RGDRGSL | 2114 |
| SNASG | 1316 | RGDRGYL | 2115 |
| SNASG | 1317 | RGDRQAL | 2116 |
| SNASG | 1318 | RGDRQSL | 2117 |
| SNASG | 1319 | RGDRQYL | 2118 |
| SNASI | 1320 | RGDRQLL | 2119 |
| SNASM | 1321 | RGDRAAL | 2120 |
| SNASM | 1322 | RGDRASL | 2121 |
| SNASM | 1323 | RGDRGAL | 2122 |
| SNASM | 1324 | RGDRGSL | 2123 |
| SNASM | 1325 | RGDRQAL | 2124 |
| SNASM | 1326 | RGDRQSL | 2125 |
| SNASS | 1327 | RGDRAAL | 2126 |
| SNASS | 1328 | RGDRASL | 2127 |
| SNASS | 1329 | RGDRAYL | 2128 |
| SNASS | 1330 | RGDRGAL | 2129 |
| SNASS | 1331 | RGDRGSL | 2130 |
| SNASS | 1332 | RGDRQAL | 2131 |
| SNASS | 1333 | RGDRQSL | 2132 |
| SNASS | 1334 | RGDRQYL | 2133 |
| SNAST | 1335 | RGDRAAL | 2134 |
| SNAST | 1336 | RGDRASL | 2135 |
| SNAST | 1337 | RGDRAYL | 2136 |
| SNAST | 1338 | RGDRGAL | 2137 |
| SNAST | 1339 | RGDRGSL | 2138 |
| SNAST | 1340 | RGDRGYL | 2139 |
| SNAST | 1341 | RGDRQAL | 2140 |
| SNAST | 1342 | RGDRQSL | 2141 |
| SNAST | 1343 | RGDRQYL | 2142 |
| SNASV | 1344 | RGDRAAL | 2143 |
| SNASV | 1345 | RGDRASL | 2144 |
| SNASV | 1346 | RGDRAYL | 2145 |
| SNASV | 1347 | RGDRGSL | 2146 |
| SNASV | 1348 | RGDRGYL | 2147 |
| SNASV | 1349 | RGDRQAL | 2148 |
| SNASV | 1350 | RGDRQSL | 2149 |
| SNASV | 1351 | RGDRQYL | 2150 |
| SNGNF | 1352 | RGDRQNL | 2151 |
| SNGNV | 1353 | RGDRGML | 2152 |
| SNGSA | 1354 | RGDRGYL | 2153 |
| SNGSA | 1355 | RGDRQAL | 2154 |
| SNGSA | 1356 | RGDRQSL | 2155 |
| SNGSA | 1357 | RGDRQYL | 2156 |
| SNGSG | 1358 | RGDRASL | 2157 |
| SNGSG | 1359 | RGDRAYL | 2158 |
| SNGSG | 1360 | RGDRGAL | 2159 |
| SNGSG | 1361 | RGDRGSL | 2160 |
| SNGSG | 1362 | RGDRGYL | 2161 |
| SNGSG | 1363 | RGDRQAL | 2162 |
| SNGSG | 1364 | RGDRQSL | 2163 |
| SNGSG | 1365 | RGDRQYL | 2164 |
| SNGSM | 1366 | RGDRAYL | 2165 |
| SNGSM | 1367 | RGDRGYL | 2166 |
| SNGSM | 1368 | RGDRQAL | 2167 |
| SNGSM | 1369 | RGDRQSL | 2168 |
| SNGSM | 1370 | RGDRQYL | 2169 |
| SNGSS | 1371 | RGDRGYL | 2170 |
| SNGSS | 1372 | RGDRQYL | 2171 |
| SNGST | 1373 | RGDRAAL | 2172 |
| SNGST | 1374 | RGDRASL | 2173 |
| SNGST | 1375 | RGDRGAL | 2174 |
| SNGST | 1376 | RGDRGYL | 2175 |
| SNGST | 1377 | RGDRQAL | 2176 |
| SNGST | 1378 | RGDRQSL | 2177 |
| SNGST | 1379 | RGDRQYL | 2178 |
| SNGSV | 1380 | RGDRGYL | 2179 |
| SNGSV | 1381 | RGDRQAL | 2180 |
| SNGSV | 1382 | RGDRQSL | 2181 |
| SNGSV | 1383 | RGDRQYL | 2182 |
| SNSGM | 1384 | RGDRGAL | 2183 |
| SNSSA | 1385 | RGDRAYL | 2184 |
| SNSSA | 1386 | RGDRGAL | 2185 |
| SNSSA | 1387 | RGDRGIL | 2186 |
| SNSSA | 1388 | RGDRGSL | 2187 |
| SNSSA | 1389 | RGDRGYL | 2188 |
| SNSSA | 1390 | RGDRQAL | 2189 |
| SNSSA | 1391 | RGDRQSL | 2190 |
| SNSSA | 1392 | RGDRQYL | 2191 |
| SNSSG | 1393 | RGDRAAL | 2192 |
| SNSSG | 1394 | RGDRAYL | 2193 |
| SNSSG | 1395 | RGDRGAL | 2194 |
| SNSSG | 1396 | RGDRGSL | 2195 |
| SNSSG | 1397 | RGDRGYL | 2196 |
| SNSSG | 1398 | RGDRQAL | 2197 |
| SNSSG | 1399 | RGDRQSL | 2198 |
| SNSSG | 1400 | RGDRQYL | 2199 |
| SNSSM | 1401 | RGDRAAL | 2200 |
| SNSSM | 1402 | RGDRASL | 2201 |
| SNSSM | 1403 | RGDRAYL | 2202 |
| SNSSM | 1404 | RGDRGSL | 2203 |
| SNSSM | 1405 | RGDRQAL | 2204 |
| SNSSM | 1406 | RGDRQSL | 2205 |
| SNSSM | 1407 | RGDRQYL | 2206 |
| SNSSS | 1408 | RGDRAAL | 2207 |
| SNSSS | 1409 | RGDRGSL | 2208 |
| SNSSS | 1410 | RGDRQAL | 2209 |
| SNSSS | 1411 | RGDRQSL | 2210 |
| SNSSS | 1412 | RGDRQYL | 2211 |
| SNSST | 1413 | RGDRAAL | 2212 |
| SNSST | 1414 | RGDRASL | 2213 |
| SNSST | 1415 | RGDRGSL | 2214 |
| SNSST | 1416 | RGDRGYL | 2215 |
| SNSST | 1417 | RGDRQAL | 2216 |
| SNSST | 1418 | RGDRQSL | 2217 |
| SNSST | 1419 | RGDRQYL | 2218 |
| SNSSV | 1420 | RGDRQAL | 2219 |
| SNSSV | 1421 | RGDRQSL | 2220 |
| SNSSV | 1422 | RGDRQYL | 2221 |
| SNTSA | 1423 | RGDRAAL | 2222 |
| SNTSA | 1424 | RGDRGAL | 2223 |
| SNTSA | 1425 | RGDRGSL | 2224 |
| SNTSA | 1426 | RGDRGYL | 2225 |
| SNTSA | 1427 | RGDRQAL | 2226 |
| SNTSA | 1428 | RGDRQSL | 2227 |
| SNTSA | 1429 | RGDRQYL | 2228 |
| SNTSG | 1430 | RGDRAAL | 2229 |
| SNTSG | 1431 | RGDRASL | 2230 |
| SNTSG | 1432 | RGDRGAL | 2231 |
| SNTSG | 1433 | RGDRGSL | 2232 |
| SNTSG | 1434 | RGDRGYL | 2233 |
| SNTSG | 1435 | RGDRQAL | 2234 |
| SNTSG | 1436 | RGDRQSL | 2235 |
| SNTSG | 1437 | RGDRQYL | 2236 |
| SNTSM | 1438 | RGDRAYL | 2237 |
| SNTSM | 1439 | RGDRGSL | 2238 |
| SNTSM | 1440 | RGDRQAL | 2239 |
| SNTSM | 1441 | RGDRQSL | 2240 |
| SNTSS | 1442 | RGDRQAL | 2241 |
| SNTST | 1443 | RGDRQAL | 2242 |
| SNTST | 1444 | RGDRQSL | 2243 |
| SNTST | 1445 | RGDRQYL | 2244 |
| SNTSV | 1446 | RGDRAYL | 2245 |
| SNTSV | 1447 | RGDRQAL | 2246 |
| SNTSV | 1448 | RGDRQSL | 2247 |
| SNTSV | 1449 | RGDRQYL | 2248 |
| SNYPH | 1450 | RGDRGAL | 2249 |
| SNYSA | 1451 | RGDRAAL | 2250 |
| SNYSA | 1452 | RGDRASL | 2251 |
| SNYSA | 1453 | RGDRGAL | 2252 |
| SNYSA | 1454 | RGDRGSL | 2253 |
| SNYSA | 1455 | RGDRQAL | 2254 |
| SNYSA | 1456 | RGDRQSL | 2255 |
| SNYSA | 1457 | RGDRQYL | 2256 |
| SNYSG | 1458 | RGDRAAL | 2257 |
| SNYSG | 1459 | RGDRGAL | 2258 |
| SNYSG | 1460 | RGDRGSL | 2259 |
| SNYSG | 1461 | RGDRQAL | 2260 |
| SNYSG | 1462 | RGDRQSL | 2261 |
| SNYSM | 1463 | RGDRAAL | 2262 |
| SNYSM | 1464 | RGDRASL | 2263 |
| SNYSM | 1465 | RGDRGSL | 2264 |
| SNYSM | 1466 | RGDRQAL | 2265 |
| SNYSM | 1467 | RGDRQSL | 2266 |
| SNYSS | 1468 | RGDRGAL | 2267 |
| SNYSS | 1469 | RGDRGSL | 2268 |
| SNYSS | 1470 | RGDRQAL | 2269 |
| SNYSS | 1471 | RGDRQSL | 2270 |
| SNYST | 1472 | RGDRAAL | 2271 |
| SNYST | 1473 | RGDRGAL | 2272 |
| SNYST | 1474 | RGDRGSL | 2273 |
| SNYST | 1475 | RGDRQAL | 2274 |
| SNYST | 1476 | RGDRQSL | 2275 |
| SNYST | 1477 | RGDRQYL | 2276 |
| SNYSV | 1478 | RGDRQAL | 2277 |
| SNYSV | 1479 | RGDRQSL | 2278 |
| SNYSV | 1480 | RGDRQYL | 2279 |
| TNSGM | 1481 | RGDRGAL | 2280 |
| VNASA | 1482 | RGDRAAL | 2281 |
| VNASA | 1483 | RGDRGAL | 2282 |
| VNASA | 1484 | RGDRGSL | 2283 |
| VNASA | 1485 | RGDRGYL | 2284 |
| VNASA | 1486 | RGDRQAL | 2285 |
| VNASA | 1487 | RGDRQYL | 2286 |
| VNASG | 1488 | RGDRAAL | 2287 |
| VNASG | 1489 | RGDRAYL | 2288 |
| VNASG | 1490 | RGDRGAL | 2289 |
| VNASG | 1491 | RGDRGSL | 2290 |
| VNASG | 1492 | RGDRGYL | 2291 |
| VNASG | 1493 | RGDRQAL | 2292 |
| VNASG | 1494 | RGDRQYL | 2293 |
| VNASM | 1495 | RGDRAYL | 2294 |
| VNASM | 1496 | RGDRQAL | 2295 |
| VNASM | 1497 | RGDRQSL | 2296 |
| VNASS | 1498 | RGDRGSL | 2297 |
| VNASS | 1499 | RGDRQAL | 2298 |
| VNASS | 1500 | RGDRQYL | 2299 |
| VNAST | 1501 | RGDRAAL | 2300 |
| VNAST | 1502 | RGDRGAL | 2301 |
| VNAST | 1503 | RGDRGSL | 2302 |
| VNAST | 1504 | RGDRGYL | 2303 |
| VNAST | 1505 | RGDRQAL | 2304 |
| VNAST | 1506 | RGDRQSL | 2305 |
| VNAST | 1507 | RGDRQYL | 2306 |
| VNASV | 1508 | RGDRGSL | 2307 |
| VNASV | 1509 | RGDRGYL | 2308 |
| VNASV | 1510 | RGDRQAL | 2309 |
| VNASV | 1511 | RGDRQSL | 2310 |
| VNASV | 1512 | RGDRQYL | 2311 |
| VNGNF | 1513 | RGDRQNL | 2312 |
| VNGNV | 1514 | RGDRGML | 2313 |
| VNGSA | 1515 | RGDRAYL | 2314 |
| VNGSA | 1516 | RGDRGAL | 2315 |
| VNGSA | 1517 | RGDRGSL | 2316 |
| VNGSA | 1518 | RGDRGYL | 2317 |
| VNGSA | 1519 | RGDRQAL | 2318 |
| VNGSA | 1520 | RGDRQSL | 2319 |
| VNGSA | 1521 | RGDRQYL | 2320 |
| VNGSG | 1522 | RGDRAAL | 2321 |
| VNGSG | 1523 | RGDRASL | 2322 |
| VNGSG | 1524 | RGDRAYL | 2323 |
| VNGSG | 1525 | RGDRGAL | 2324 |
| VNGSG | 1526 | RGDRGSL | 2325 |
| VNGSG | 1527 | RGDRGYL | 2326 |
| VNGSG | 1528 | RGDRQAL | 2327 |
| VNGSG | 1529 | RGDRQSL | 2328 |
| VNGSG | 1530 | RGDRQYL | 2329 |
| VNGSM | 1531 | RGDRGYL | 2330 |
| VNGSM | 1532 | RGDRQAL | 2331 |
| VNGSM | 1533 | RGDRQSL | 2332 |
| VNGSM | 1534 | RGDRQYL | 2333 |
| VNGSS | 1535 | RGDRAYL | 2334 |
| VNGSS | 1536 | RGDRGAL | 2335 |
| VNGSS | 1537 | RGDRGSL | 2336 |
| VNGSS | 1538 | RGDRQAL | 2337 |
| VNGSS | 1539 | RGDRQSL | 2338 |
| VNGSS | 1540 | RGDRQYL | 2339 |
| VNGST | 1541 | RGDRAAL | 2340 |
| VNGST | 1542 | RGDRAYL | 2341 |
| VNGST | 1543 | RGDRGAL | 2342 |
| VNGST | 1544 | RGDRGYL | 2343 |
| VNGST | 1545 | RGDRQAL | 2344 |
| VNGST | 1546 | RGDRQSL | 2345 |
| VNGST | 1547 | RGDRQYL | 2346 |
| VNGSV | 1548 | RGDRAYL | 2347 |
| VNGSV | 1549 | RGDRQAL | 2348 |
| VNGSV | 1550 | RGDRQSL | 2349 |
| VNGSV | 1551 | RGDRQYL | 2350 |
| VNSGM | 1552 | RGDRGAL | 2351 |
| VNSSA | 1553 | RGDRGAL | 2352 |
| VNSSA | 1554 | RGDRGSL | 2353 |
| VNSSA | 1555 | RGDRGYL | 2354 |
| VNSSA | 1556 | RGDRQAL | 2355 |
| VNSSA | 1557 | RGDRQSL | 2356 |
| VNSSA | 1558 | RGDRQYL | 2357 |
| VNSSG | 1559 | RGDRAAL | 2358 |
| VNSSG | 1560 | RGDRAYL | 2359 |
| VNSSG | 1561 | RGDRGAL | 2360 |
| VNSSG | 1562 | RGDRQAL | 2361 |
| VNSSG | 1563 | RGDRQSL | 2362 |
| VNSSG | 1564 | RGDRQYL | 2363 |
| VNSSM | 1565 | RGDRAYL | 2364 |
| VNSSM | 1566 | RGDRQAL | 2365 |
| VNSSM | 1567 | RGDRQSL | 2366 |
| VNSSM | 1568 | RGDRQYL | 2367 |
| VNSSS | 1569 | RGDRGSL | 2368 |
| VNSSS | 1570 | RGDRQAL | 2369 |
| VNSSS | 1571 | RGDRQSL | 2370 |
| VNSST | 1572 | RGDRGAL | 2371 |
| VNSST | 1573 | RGDRQAL | 2372 |
| VNSST | 1574 | RGDRQSL | 2373 |
| VNSST | 1575 | RGDRQYL | 2374 |
| VNSSV | 1576 | RGDRAYL | 2375 |
| VNSSV | 1577 | RGDRQAL | 2376 |
| VNSSV | 1578 | RGDRQSL | 2377 |
| VNTSA | 1579 | RGDRAYL | 2378 |
| VNTSG | 1580 | RGDRAAL | 2379 |
| VNTSG | 1581 | RGDRGAL | 2380 |
| VNTSG | 1582 | RGDRGSL | 2381 |
| VNTSG | 1583 | RGDRGYL | 2382 |
| VNTSG | 1584 | RGDRQAL | 2383 |
| VNTSG | 1585 | RGDRQSL | 2384 |
| VNTSG | 1586 | RGDRQYL | 2385 |
| VNTST | 1587 | RGDRAYL | 2386 |
| VNYPH | 1588 | RGDRGAL | 2387 |
| VNYSA | 1589 | RGDRGAL | 2388 |
| VNYSA | 1590 | RGDRGSL | 2389 |
| VNYSA | 1591 | RGDRQAL | 2390 |
| VNYSA | 1592 | RGDRQSL | 2391 |
| VNYSG | 1593 | RGDRAAL | 2392 |
| VNYSG | 1594 | RGDRGAL | 2393 |
| VNYSG | 1595 | RGDRQAL | 2394 |
| VNYSG | 1596 | RGDRQSL | 2395 |
| VNYSG | 1597 | RGDRQYL | 2396 |
| VNYSS | 1598 | RGDRGSL | 2397 |
| VNYSS | 1599 | RGDRGYL | 2398 |

### BRIEF DESCRIPTION OF THE DRAWINGS

FIG. 1 is a schematic of the novel platform of the invention.

FIG. 2 is a schematic of the novel capsid platform of the invention.

FIG. 3A show images of fluorescence in tissues following administration of capsids of the invention.

FIG. 3B shows graphs of transgene mRNA in tissues following administration of capsids of the invention.

FIG. 4A-B show graphs of relative luminescence in mouse and human primary myotubules following administration of capsids of the invention.

FIG. 5A-5B shows graphs of transduction percentage in mouse and human primary myotubules following administration of capsids of the invention.

FIG. 6 is a graph of probability of survival following capsid and transgene administration in Mtm1 knockout mice.

FIG. 7 is an image of MTM1 levels in non-human primate skeletal muscle following capsid and transgene administration.

FIG. 8 shows images of fluorescence in tissues following administration of capsids of the invention.

FIG. 9A-B show mRNA transgene expression levels in NHP skeletal muscle following administration of capsids of the invention.

FIG. 10A-B show mRNA transgene expression levels in NHP cardiac muscle following administration of capsids of the invention.

FIG. 11A-B show mRNA transgene expression levels in NHP liver following administration of capsids of the invention.

FIG. 12A-B show mRNA transgene expression levels in NHP dorsal root ganglia (DRG) following administration of capsids of the invention.

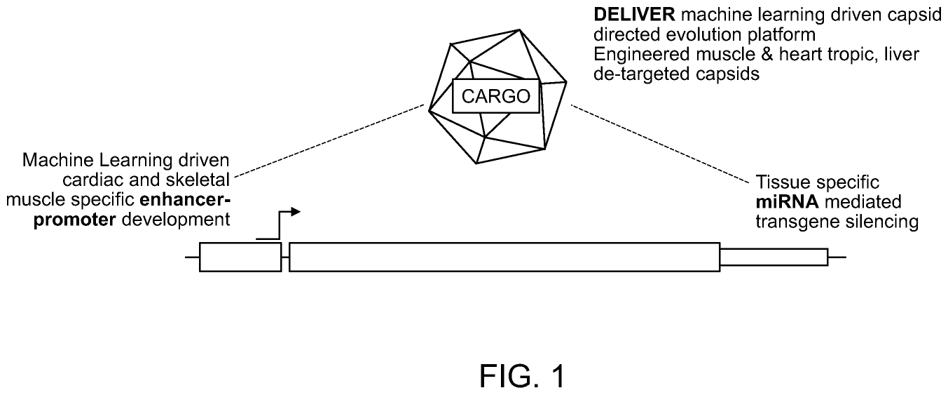

FIG. 1-25 are Appendix A providing results from capsids of the invention (MyoAAV-LD 6).

### DETAILED DESCRIPTION

The present invention provides novel capsid protein variants for viral vectors that detarget liver tissue and target skeletal muscle and heart tissue at the same time. Aspects of the present invention provide adeno-associated virus (AAV) vector's comprising a capsid protein comprising the amino acid sequence RGDR. In the capsid protein, RGDR may be inserted after amino acid 455 in reference to an AAV9 capsid or equivalent position in another AAV capsid. For example, capsid proteins variants of the invention may comprise a sequence as set forth in Table 1.

### Adeno Associated Virus Vectors

AAVs are particularly appropriate viral vectors for delivery of genetic material into mammalian cells. AAVs are not known to cause disease in mammals and cause a very mild immune response. Additionally, AAVs are able to infect cells in multiple stages whether at rest or in a phase of the cell replication cycle. Advantageously, AAV DNA is not regularly inserted into the host's genome at random sites, reducing the oncogenic properties of this vector.

AAVs have been engineered to deliver a variety of treatments, especially for genetic disorders caused by single nucleotide polymorphisms (“SNP”). Genetic diseases that have been studied in conjunction with AAV vectors include Cystic fibrosis, hemophilia, arthritis, macular degeneration, muscular dystrophy, Parkinson's disease, congestive heart failure, and Alzheimer's disease. The AAV can be used as a vector to deliver engineered nucleic acid to a host and utilize the host's own ribosomes to transcribe that nucleic acid into the desired proteins. See, e.g., West et al., Virology 160:38-47 (1987); U.S. Pat. No. 4,797,368; WO 93/24641; Kotin, Human Gene Therapy 5:793-801 (1994); and Muzyczka, J. Clin. Invest. 94:1351 (1994). AAVs have some deficiency in their replication and/or pathogenicity and thus can be safer that adenoviral vectors. In some embodiments, the AAV can integrate into a specific site on chromosome 19 of a human cell with no observable side effects. In some embodiments, the capacity of the AAV vector, system thereof, and/or AAV particles can be up to about 4.7 kb. The AAV vector or system thereof can include one or more engineered capsid polynucleotides described herein.

AAVs are small, replication-defective, nonenveloped viruses that infect humans and other primate species and have a linear single-stranded DNA genome. Naturally occurring AAV serotypes exhibit liver tropism. As a result, transfection of non-liver tissue with traditional AAV vectors is impeded by the virus's natural liver tropism. Moreover, because the liver acts to break down substances delivered to a subject, transfection of non-liver tissue with unmodified AAV vectors requires higher dosing to provide sufficient viral load to overcome the liver and reach non-liver tissue. More than 30 naturally occurring serotypes of AAV are available. Many natural variants in the AAV capsid exist. AAV serotypes include, but are not limited to, AAV serotypes AAV1, AAV2, AAV3, AAV3B, AAV4, AAV5, AAV6, AAV7, AAV8, AAV9, AAV10, AAV11, AAV 12, AAV13. AAVs may be engineered using conventional molecular biology techniques, making it possible to optimize these particles, for example, for cell specific delivery, for minimizing immunogenicity, for tuning stability and particle lifetime, for efficient degradation, for accurate delivery to the nucleus. AAV vectors can be specifically targeted to one or more types of cells by choosing the appropriate combination of AAV serotype, promoter, and delivery method.

Previous approaches to identify AAV sequences correlated with tropism have relied upon the comparison of highly related extant serotypes with distinct characteristics, random domain swaps between unrelated serotypes, or consideration of higher-order structure, to identify motifs that define liver tropism. For example, mapping determinants of AAV tropism have been carried out by comparing highly related serotypes. One such example is the single-amino acid change (E531K) between AAV1 and AAV6 that improves murine liver transduction in AAV1. See Wu et al. (2006) J. Virol., 80 (22): 11393-7, incorporated by reference herein. Another example is a reciprocal domain swap between AAV2 and AAV8 that alters tropism, but fails to define any robust specific tissue-targeting motifs. See Raupp et al. (201) J. Virol., 86 (17): 9396-408, incorporated by reference herein. Further, global consideration of structure has only highlighted gross differences between better- or worse-liver-transducers that are more observational than useful in practice. Nam et al (2007) J. Virol., 81 (22): 12260-71.

AAVs exhibiting modified tissue tropism that may be used with the present invention are described in U.S. Pat. Nos. 9,695,220, 9,719,070; 10,119,125; 10,526,584; U.S. Patent Application Publication No. 2018-0369414; U.S. Patent Application Publication No. 2020-0123504; U.S. Patent Application Publication No. 2020-0318082; PCT International Patent Application Publication No. WO 2015/054653; PCT International Patent Application Publication No. WO 2016/179496; PCT International Patent Application Publication No. WO 2017/100791; and PCT International Patent Application Publication No. WO 2019/217911, the entirety of the contents of each of which are incorporated by reference herein.

The AAV vector or system thereof may include one or more regulatory molecules, such as promoters, enhancers, repressors and the like. In some embodiments, the AAV vector or system thereof can include one or more polynucleotides that can encode one or more regulatory proteins. In some embodiments, the one or more regulatory proteins can be selected from Rep78, Rep68, Rep52, Rep40, variants thereof, and combinations thereof. In some embodiments, the muscle specific promoter can drive expression of an engineered AAV capsid polynucleotide.

The AAV vector or system thereof can include one or more polynucleotides that can encode one or more capsid proteins, such as the engineered AAV capsid proteins described elsewhere herein. The engineered capsid proteins can be capable of assembling into a protein shell (an engineered capsid) of the AAV virus particle. The engineered capsid can have a cell-, tissue-, and/or organ-specific tropism.

The AAV vector or system thereof can be configured to produce AAV particles having a specific serotype. In some embodiments, the serotype can be AAV-1, AAV-2, AAV-3, AAV-4, AAV-5, AAV-6, AAV-8, AAV-9 or any combinations thereof. In some embodiments, the AAV can be AAV1, AAV-2, AAV-5, AAV-9 or any combination thereof. One can select the AAV of the AAV with regard to the cells to be targeted; e.g., one can select AAV serotypes 1, 2, 5, 9 or a hybrid capsid AAV-1, AAV-2, AAV-5, AAV-9 or any combination thereof for targeting brain and/or neuronal cells; and one can select AAV-4 for targeting cardiac tissue; and one can select AAV-8 for delivery to the liver. Thus, in some embodiments, an AAV vector or system thereof capable of producing AAV particles capable of targeting the brain and/or neuronal cells can be configured to generate AAV particles having serotypes 1, 2, 5 or a hybrid capsid AAV-1, AAV-2, AAV-5 or any combination thereof. In some embodiments, an AAV vector or system thereof capable of producing AAV particles capable of targeting cardiac tissue can be configured to generate an AAV particle having an AAV-4 serotype. In some embodiments, an AAV vector or system thereof capable of producing AAV particles capable of targeting the liver can be configured to generate an AAV having an AAV-8 serotype. See also Srivastava. 2017. Curr. Opin. Virol. 21:75-80.

It will be appreciated that while the different serotypes can provide some level of cell, tissue, and/or organ specificity, each serotype still is multi-tropic and thus can result in tissue-toxicity if using that serotype to target a tissue that the serotype is less efficient in transducing. Thus, in addition to achieving some tissue targeting capacity via selecting an AAV of a particular serotype, it will be appreciated that the tropism of the AAV serotype can be modified by an engineered AAV capsid described herein. As described elsewhere herein, variants of wild-type AAV of any serotype can be generated via a method described herein and determined to have a particular cell-specific tropism, which can be the same or different as that of the reference wild-type AAV serotype. In some embodiments, the cell, tissue, and/or specificity of the wild-type serotype can be enhanced (e.g., made more selective or specific for a particular cell type that the serotype is already biased towards). For example, wild-type AAV-9 is biased towards muscle and brain in humans (see e.g., Srivastava. 2017. Curr. Opin. Virol. 21:75-80.) By including an engineered AAV capsid and/or capsid protein variant of wild-type AAV-9 as described herein, the tropism for nervous cells might be reduced or eliminated and/or the muscle specificity increased such that the nervous specificity appears reduced in comparison, thus enhancing the specificity for muscle as compared to the wild-type AAV-9. As previously mentioned, inclusion of an engineered capsid and/or capsid protein variant of a wild-type AAV serotype can have a different tropism than the wild-type reference AAV serotype. For example, an engineered AAV capsid and/or capsid protein variant of AAV-9 can have specificity for a tissue other than muscle or brain in humans.

In some embodiments, the AAV vector is a hybrid AAV vector or system thereof. Hybrid AAVs are AAVs that include genomes with elements from one serotype that are packaged into a capsid derived from at least one different serotype. For example, if it is the rAAV2/5 that is to be produced, and if the production method is based on the helper-free, transient transfection method discussed above, the 1st plasmid and the 3rd plasmid (the adeno helper plasmid) will be the same as discussed for rAAV2 production. However, the 2nd plasmid, the pRepCap will be different. In this plasmid, called pRep2/Cap5, the Rep gene is still derived from AAV2, while the Cap gene is derived from AAV5. The production scheme is the same as the above-mentioned approach for AAV2 production. The resulting rAAV is called rAAV2/5, in which the genome is based on recombinant AAV2, while the capsid is based on AAV5. It is assumed the cell or tissue-tropism displayed by this AAV2/5 hybrid virus should be the same as that of AAV5. It will be appreciated that wild-type hybrid AAV particles suffer the same specificity issues as with the non-hybrid wild-type serotypes previously discussed.

Advantages achieved by the wild-type based hybrid AAV systems can be combined with the increased and customizable cell-specificity that can be achieved with the engineered AAV capsids can be combined by generating a hybrid AAV that can include an engineered AAV capsid described elsewhere herein. It will be appreciated that hybrid AAVs can contain an engineered AAV capsid containing a genome with elements from a different serotype than the reference wild-type serotype that the engineered AAV capsid is a variant of. For example, a hybrid AAV can be produced that includes an engineered AAV capsid that is a variant of an AAV-9 serotype that is used to package a genome that contains components (e.g., rep elements) from an AAV-2 serotype. As with wild-type based hybrid AAVs previously discussed, the tropism of the resulting AAV particle will be that of the engineered AAV capsid.

In some embodiments, the AAV vector or system thereof is configured as a “gutless” vector, similar to that described in connection with a retroviral vector. In some embodiments, the “gutless” AAV vector or system thereof can have the cis-acting viral DNA elements involved in genome amplification and packaging in linkage with the heterologous sequences of interest (e.g., the engineered AAV capsid polynucleotide(s)).

The vectors described herein can be constructed using any suitable process or technique. In some embodiments, one or more suitable recombination and/or cloning methods or techniques can be used to the vector(s) described herein. Suitable recombination and/or cloning techniques and/or methods can include, but not limited to, those described in U.S. Application publication No. US 2004-0171156 A1. Other suitable methods and techniques are described elsewhere herein.

Construction of recombinant AAV vectors are described in a number of publications, including U.S. Pat. No. 5,173,414; Tratschin et al., Mol. Cell. Biol. 5:3251-3260 (1985); Tratschin, et al., Mol. Cell. Biol. 4:2072-2081 (1984); Hermonat & Muzyczka, PNAS 81:6466-6470 (1984); and Samulski et al., J. Virol. 63:03822-3828 (1989). Any of the techniques and/or methods can be used and/or adapted for constructing an AAV or other vector described herein. AAV vectors are discussed elsewhere herein.

In some embodiments, the vector can have one or more insertion sites, such as a restriction endonuclease recognition sequence (also referred to as a “cloning site”). In some embodiments, one or more insertion sites (e.g., about or more than about 1, 2, 3, 4, 5, 6, 7, 8, 9, 10, or more insertion sites) are located upstream and/or downstream of one or more sequence elements of one or more vectors.

Delivery vehicles, vectors, particles, nanoparticles, formulations and components thereof for expression of one or more elements of a engineered AAV capsid system described herein are as used in the foregoing documents, such as International Patent Application Publications WO WO 2021/050974 and WO 2021/077000 and PCT International Application No. PCT/US2021/042812, the contents of which are incorporated by reference herein.

Additional AAV vectors are described in International Patent Application Publication WO 2019/2071632, the contents of which are incorporated by reference herein.

Further AAV vectors are described in International Patent Application Publications WO 2020/086881 and WO 2020/235543, the contents of each of which are incorporated by reference herein.

Further AAV vectors are described in International Patent Application Publications WO 2005/033321; WO 2006/110689; WO 2007/127264; WO 2008/027084; WO 2009/073103; WO 2009/073104; WO 2009/105084; WO 2009/134681; WO 2009/136977; WO 2010/051367; WO 2010/138675; WO 2001/038187; WO 2012/112832; WO 2015/054653; WO 2016/179496; WO 2017/100791; WO 2017/019994; WO 2018/209154; WO 2019/067982; WO 2019/195701; WO 2019/217911; WO 2020/041498; WO 2020/210839; U.S. Pat. Nos. 7,906,111; 9,737,618; 10,265,417; 10,485,883; 10,695,441; 10,722,598; 8,999,678; 10,301,648; 10,626,415; 9,198,984; 10,155,931; 8,524,219; 9,206,238; 8,685,387; 9,359,618; 8,231,880; 8,470,310; 9,597,363; 8,940,290; 9,593,346; 10,501,757; 10,786,568; 10,973,928; 10,519,198; 8,846,031; 9,617,561; 9,884,071; 10,406,173; 9,596,220; 9,719,010; 10,117,125; 10,526,584; 10,881,548; 10,738,087; U.S. Patent Publication No. 2011-023353; U.S. Patent Publication No. 2019-0015527; U.S. Patent Publication No. 2020-155704; U.S. Patent Publication No 2017-0191079; U.S. Patent Publication No. 2019-0218574; U.S. Patent Publication No. 2020-0208176; U.S. Patent Publication No. 2020-0325491; U.S. Patent Publication No. 2019-0055523; U.S. Patent Publication No. 2020-0385689; U.S. Patent Publication No. 2009-0317417; U.S. Patent Publication No. 2016-0051603; U.S. Patent Publication No. 2016-00244783; U.S. Patent Publication No. 2017-0183636; U.S. Patent Publication No. 2020-0263201; U.S. Patent Publication No. 2020-0101099; U.S. Patent Publication No. 2020-0318082; U.S. Patent Publication No. 2018-0369414; U.S. Patent Publication No. 2019-0330278; U.S. Patent Publication No. 2020-0231986, the contents of each of which are incorporated by reference herein.

### Capsid Protein

The capsid protein is the shell or coating of the virus that enables its delivery into the host. Without the protein, the nucleic acids would be destroyed by the host without entering into the host cells and beginning transcription and translation. The capsid protein may be in the natural conformation of a naturally occurring AAV, or it may be modified.

In certain example embodiments, the AAV capsid protein is an engineered AAV capsid protein having reduced or eliminated uptake in a non-muscle cell as compared to a corresponding wild-type AAV capsid polypeptide.

In some embodiments, the engineered AAV capsid encoding polynucleotide can be included in a polynucleotide that is configured to be an AAV genome donor in an AAV vector system that can be used to generate engineered AAV particles described elsewhere herein. In some embodiments, the engineered AAV capsid encoding polynucleotide can be operably coupled to a poly adenylation tail. In some embodiments, the poly adenylation tail can be an SV40 poly adenylation tail. In some embodiments, the AAV capsid encoding polynucleotide can be operably coupled to a promoter. In some embodiments, the promoter can be a tissue specific promoter. In some embodiments, the tissue specific promoter is specific for muscle (e.g., cardiac, skeletal, and/or smooth muscle), neurons and supporting cells (e.g., astrocytes, glial cells, Schwann cells, etc.), fat, spleen, liver, kidney, immune cells, spinal fluid cells, synovial fluid cells, skin cells, cartilage, tendons, connective tissue, bone, pancreas, adrenal gland, blood cell, bone marrow cells, placenta, endothelial cells, and combinations thereof. In some embodiments, the promoter can be a constitutive promoter. Suitable tissue specific promoters and constitutive promoters are discussed elsewhere herein and are generally known in the art and can be commercially available. Suitable muscle specific promoters include, but are not limited to CK8, MHCK7, Myoglobin promoter (Mb), Desmin promoter, muscle creatine kinase promoter (MCK) and variants thereof, and SPc5-12 synthetic promoter.

Described herein are various embodiments of engineered viral capsids, such as adeno-associated virus (AAV) capsids, that can be engineered to confer cell-specific tropism, such as muscle specific tropism, to an engineered viral particle. Engineered viral capsids can be lentiviral, retroviral, adenoviral, or AAV capsids. The engineered capsids can be included in an engineered virus particle (e.g., an engineered lentiviral, retroviral, adenoviral, or AAV virus particle), and can confer cell-specific tropism, reduced immunogenicity, or both to the engineered viral particle. The engineered viral capsids described herein can include one or more engineered viral capsid proteins described herein. The engineered viral capsids described herein can include one or more engineered viral capsid proteins described herein that can contain a muscle-specific targeting moiety containing or composed of an n-mer motif described elsewhere herein.

The engineered viral capsid and/or capsid proteins can be encoded by one or more engineered viral capsid polynucleotides. In some embodiments, the engineered viral capsid polynucleotide is an engineered AAV capsid polynucleotide, engineered lentiviral capsid polynucleotide, engineered retroviral capsid polynucleotide, or engineered adenovirus capsid polynucleotide. In some embodiments, an engineered viral capsid polynucleotide (e.g., an engineered AAV capsid polynucleotide, engineered lentiviral capsid polynucleotide, engineered retroviral capsid polynucleotide, or engineered adenovirus capsid polynucleotide) can include a 3′ polyadenylation signal. The polyadenylation signal can be an SV40 polyadenylation signal.

The engineered viral capsids can be variants of wild-type viral capsid. For example, in some embodiments, the engineered AAV capsids can be variants of wild-type AAV capsids. In some embodiments, the wild-type AAV capsids can be composed of VP1, VP2, VP3 capsid proteins or a combination thereof. In other words, the engineered AAV capsids can include one or more variants of a wild-type VP1, wild-type VP2, and/or wild-type VP3 capsid proteins. In some embodiments, the serotype of the reference wild-type AAV capsid can be AAV-1, AAV-2, AAV-3, AAV-4, AAV-5, AAV-6, AAV-8, AAV-9 or any combination thereof. In some embodiments, the serotype of the wild-type AAV capsid can be AAV-9. The engineered AAV capsids can have a different tropism than that of the reference wild-type AAV capsid.

The engineered viral capsid can contain 1-60 engineered capsid proteins. In some embodiments, the engineered viral capsids can contain 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 engineered capsid proteins. In some embodiments, the engineered viral capsid can contain 0-59 wild-type viral capsid proteins. In some embodiments, the engineered viral capsid can contain 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, or 59 wild-type viral capsid proteins.

In some embodiments, the engineered AAV capsid can contain 1-60 engineered capsid proteins. In some embodiments, the engineered AAV capsids can contain 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 engineered capsid proteins. In some embodiments, the engineered AAV capsid can contain 0-59 wild-type AAV capsid proteins. In some embodiments, the engineered AAV capsid can contain 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, or 59 wild-type AAV capsid proteins.

In some embodiments, the engineered viral capsid protein can have an n-mer amino acid motif, where n can be at least 3 amino acids. In some embodiments, n can be 3, 4, 5, 6, 7, 8, 9, 10, 11, 12, 13, 14, or 15 amino acids. In some embodiments, an engineered AAV capsid can have a 6-mer or 7-mer amino acid motif. In some embodiments, the n-mer amino acid motif can be inserted between two amino acids in the wild-type viral protein (VP) (or capsid protein). In some embodiments, the n-mer motif can be inserted between two amino acids in a variable amino acid region in a viral capsid protein.

In some embodiments, the n-mer motif can be inserted between two amino acids in a variable amino acid region in an AAV capsid protein. The core of each wild-type AAV viral protein contains an eight-stranded beta-barrel motif (betaB to betaI) and an alpha-helix (alphaA) that are conserved in autonomous parvovirus capsids (see e.g., DiMattia et al. 2012. J. Virol. 86 (12): 6947-6958). Structural variable regions (VRs) occur in the surface loops that connect the beta-strands, which cluster to produce local variations in the capsid surface. AAVs have 12 variable regions (also referred to as hypervariable regions) (see e.g., Weitzman and Linden. 2011. “Adeno-Associated Virus Biology.” In Snyder, R. O., Moullier, P. (eds.) Totowa, NJ: Humana Press). In some embodiments, one or more n-mer motifs can be inserted between two amino acids in one or more of the 12 variable regions in the wild-type AVV capsid proteins. In some embodiments, the one or more n-mer motifs can be each be inserted between two amino acids in VR-I, VR-II, VR-III, VR-IV, VR-V, VR-VI, VR-VII, VR-III, VR-IX, VR-X, VR-XI, VR-XII, or a combination thereof. In some embodiments, the n-mer can be inserted between two amino acids in the VR-III of a capsid protein. In some embodiments, the engineered capsid can have an n-mer inserted between any two contiguous amino acids between amino acids 262 and 269, between any two contiguous amino acids between amino acids 327 and 332, between any two contiguous amino acids between amino acids 382 and 386, between any two contiguous amino acids between amino acids 452 and 460, between any two contiguous amino acids between amino acids 488 and 505, between any two contiguous amino acids between amino acids 545 and 558, between any two contiguous amino acids between amino acids 581 and 593, between any two contiguous amino acids between amino acids 704 and 714 of an AAV9 viral protein. In some embodiments, the engineered capsid can have an n-mer inserted between amino acids 588 and 589 of an AAV9 viral protein. In some embodiments, the engineered capsid can have a 7-mer motif inserted between amino acids 588 and 589 of an AAV9 viral protein. In other embodiments, the motif inserted is a 10-mer motif, with replacement of amino acids 586-88 and an insertion before 589. SEQ ID NO. 1 is a reference AAV9 capsid sequence for at least referencing the insertion sites discussed above. It will be appreciated that n-mers can be inserted in analogous positions in AAV viral proteins of other serotypes. In some embodiments as previously discussed, the n-mer(s) can be inserted between any two contiguous amino acids within the AAV viral protein and in some embodiments the insertion is made in a variable region.

In some embodiments, the first 1, 2, 3, or 4 amino acids of an n-mer motif can replace 1, 2, 3, or 4 amino acids of a polypeptide into which it is inserted and preceding the insertion site. In some embodiments, the amino acids of the n-mer motif that replace 1 or more amino acids of the polypeptide into which the n-mer motif is inserted come before or immediately before an “RGD” in an n-mer motif. For example, in one or more of the 10-mer inserts, the first three amino acids shown can replace 1-3 amino acids into a polypeptide to which they may be inserted. Using an AAV as another non-limiting example, one or more of the n-mer motifs can be inserted into e.g., and AAV9 capsid prolylpeptide between amino acids 588 and 589 and the insert can replace amino acids 586, 587, and 588 such that the amino acid immediately preceding the n-mer motif after insertion is residue 585. It will be appreciated that this principle can apply in any other insertion context and is not necessarily limited to insertion between residues 588 and 589 of an AAV9 capsid or equivalent position in another AAV capsid. It will further be appreciated that in some embodiments, no amino acids in the polypeptide into which the n-mer motif is inserted are replaced by the n-mer motif.

In some embodiments, the AAV capsids or other viral capsids or compositions can be muscle-specific. In some embodiments, muscle-specificity of the engineered AAV or other viral capsid or other composition is conferred by a muscle specific n-mer motif incorporated in the engineered AAV or other viral capsid or other composition described herein. While not intending to be bound by theory, it is believed that the n-mer motif confers a 3D structure to or within a domain or region of the engineered AAV capsid or other viral capsid or other composition such that the interaction of the viral particle or other composition containing the engineered AAV capsid or other viral capsid or other composition described herein has increased or improved interactions (e.g., increased affinity) with a cell surface receptor and/or other molecule on the surface of a muscle cell. In some embodiments, the cell surface receptor is AAV receptor (AAVR). In some embodiments, the cell surface receptor is a muscle cell specific AAV receptor. In some embodiments, the cell surface receptor or other molecule is a cell surface receptor or other molecule selectively expressed on the surface of a muscle cell. In some embodiments, the cell surface receptor or molecule is an integrin or dimer thereof. In some embodiments, the cell surface receptor or molecule is an Vb6 integrin heterodimer.

In some embodiments, a muscle specific engineered viral particle or other composition described herein containing the muscle-specific capsid, n-mer motif, or muscle-specific targeting moiety described herein can have an increased uptake, delivery rate, transduction rate, efficiency, amount, or a combination thereof in a muscle cell as compared to other cells types and/or other virus particles (including but not limited to AAVs) and other compositions that do not contain the muscle-specific n-mer motif of the present invention.

First- and second-generation muscle specific AAV capsids were developed using a muscle specific promoter and the resulting capsid libraries were screened in mice and non-human primates as described elsewhere herein and/or in e.g., U.S. Provisional Application Ser. Nos. 62/899,453, 62/916,207, 63/018,454, 63/242,008, and No. 63/345,14.

### EXPERIMENTAL DETAILS

Co-evolution of liver de-targeting and muscle targeting features resulted in development of MyoAAV-LD capsids in NHPs, resulting in “6th generation” capsids terms MyoAAV-LD 6.

### First Generation Muscle Gene Therapies are not Optimized for Efficacy and Safety.

Naturally occurring AAV capsids do not transduce muscle effectively.

Capsids used in first-generation gene therapies (e.g. AAV8, AAV9, AAVrh74) are mainly sequestered in the liver after systemic administration, requiring very high doses to be effective in muscle disease.

High doses cause toxicity. Serious side effects have occurred with high dose gene therapies, including liver injury, and an acute blood disorder called thrombotic microangiopathy that can cause kidney injury.

Cargoes lack effective and selective regulatory elements. First generation AAV muscle gene therapies do not use highly optimized skeletal muscle and cardiac regulatory elements, potential for off-target toxicity.

More potent and tissue selective capsids are required to treat muscle and heart disease safely and effectively.

### Novel Capsids

A novel platform was utilized comprising a capsid and gene expression regulation technologies to address critical challenges in muscle gene therapies.

FIG. 1 is a schematic of the novel platform of the invention. Machine learning driven cardiac and skeletal muscle specific regulatory elements (enhancer-promoter) were developed. Tissue specific miRNA mediated transgene silencing was developed. Machine learning driven capsid directed evolution was utilized to engineer muscle and heart tropic, liver de-targeted capsids.

### Capsid Development

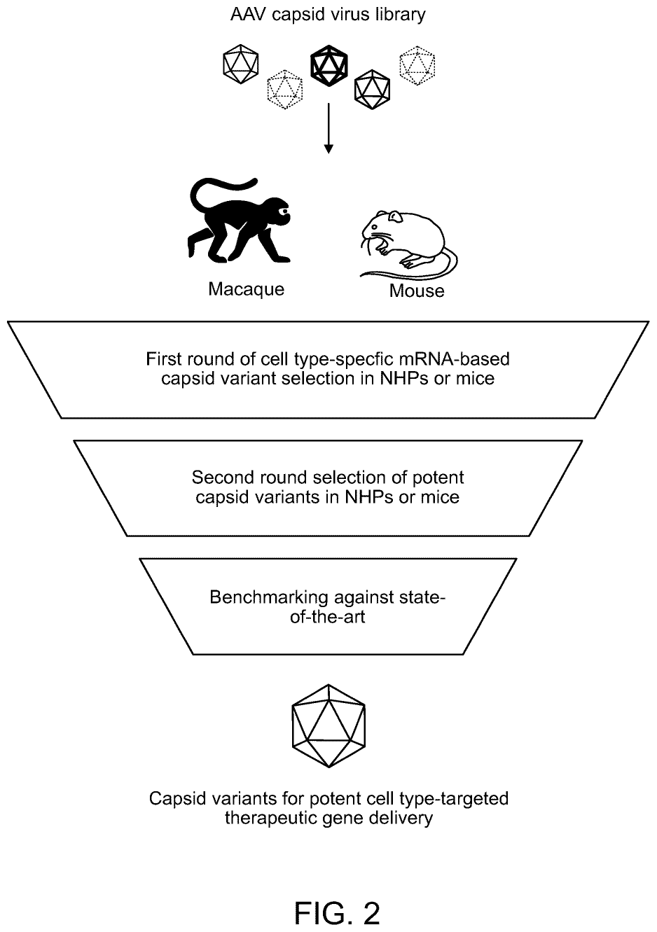

FIG. 2 is a schematic of the novel capsid platform of the invention. Directed evolution of AAV capsids leveraged in vivo expression of transgene RNA. A diverse first-round virus library was designed based on the capsid 3D structure. Machine learning-based design was used for a second-round capsid library. Stringent mRNA-based selection was conduct of capsid variants. Potent capsid variants in any specific or cell type of interest (non-human primates, mice) was conducted.

The platform identified RGD-containing MyoAAV class of skeletal muscle and cardiotropic capsids in mice and non-human primates.

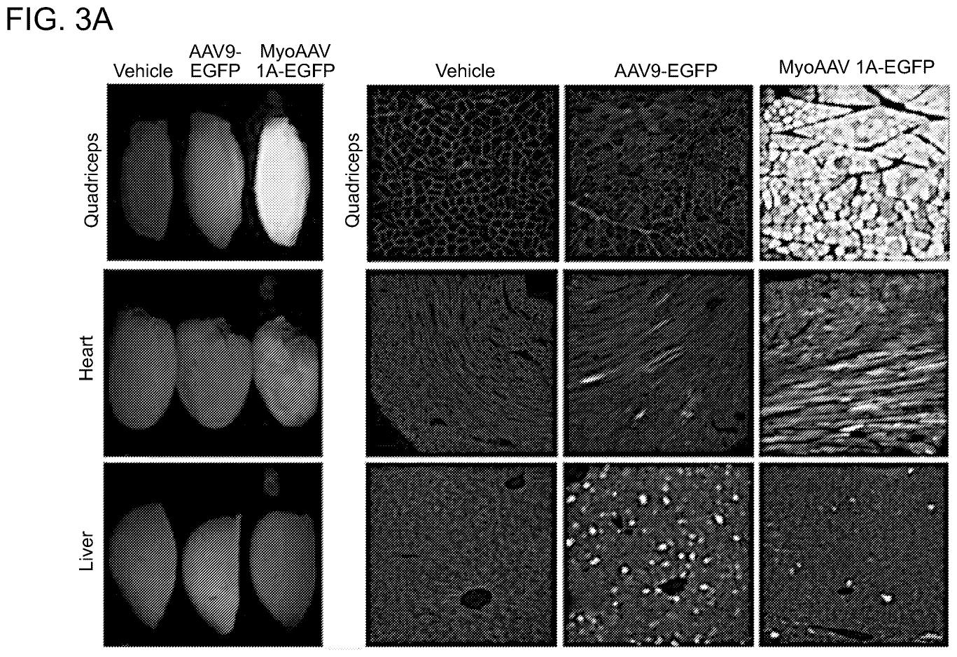

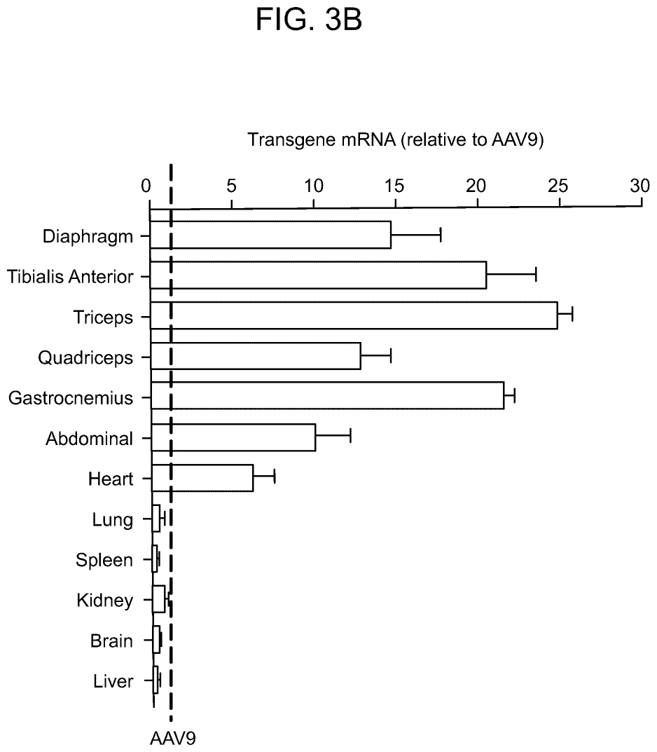

FIG. 3A shows images of fluorescence in tissues following administration of capsids of the invention.

FIG. 3B shows graphs of transgene mRNA in tissues following administration of capsids of the invention.

5E+13 vg/kg of MyoAAVIA- or AAV9-EGFP was administered to tissues harvested 14 days post IV administration from adult C57BL/6 mice. The platform was successful in identifying a class of skeletal muscle and cardiotropic capsids MyoAAV class capsids showed unprecedented targeting to skeletal muscle and heart.

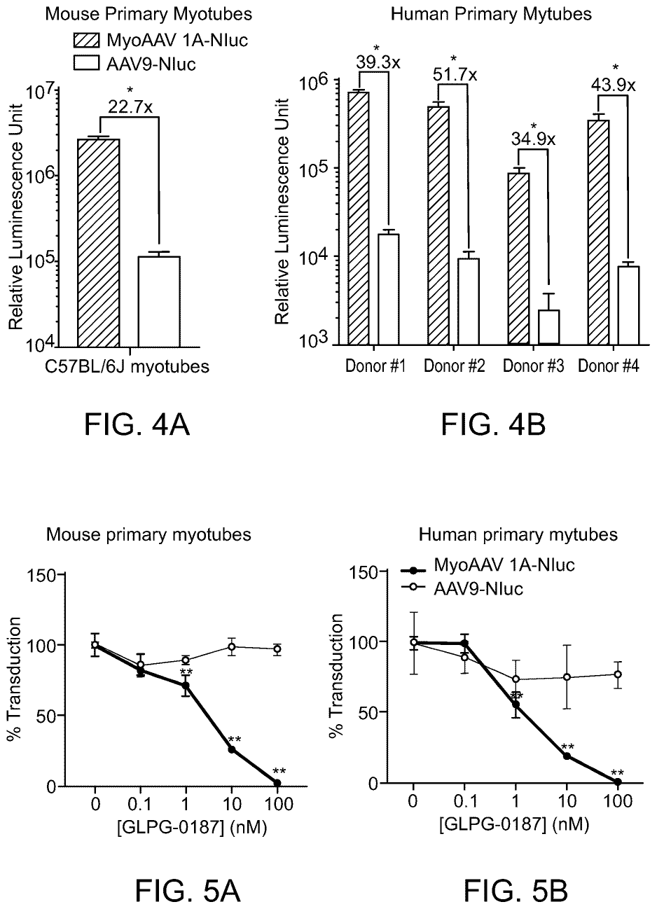

FIG. 4A-B show graphs of relative luminescence in mouse and human primary myotubules following administration of capsids of the invention.

FIG. 5A-5B shows graphs of transduction percentage in mouse and human primary myotubules following administration of capsids of the invention.

Muscle tropism of MyoAAV was conserved across species and dependent on aV-containing integrin heterodimers. The conserved mechanism of transduction provides confidence in translating potency to humans.

### Generation of MyoAAV Capsids

MyoAAV capsid generation led to a more potent muscle-tropic gene therapy candidate for the treatment of XLMTM (KT430).

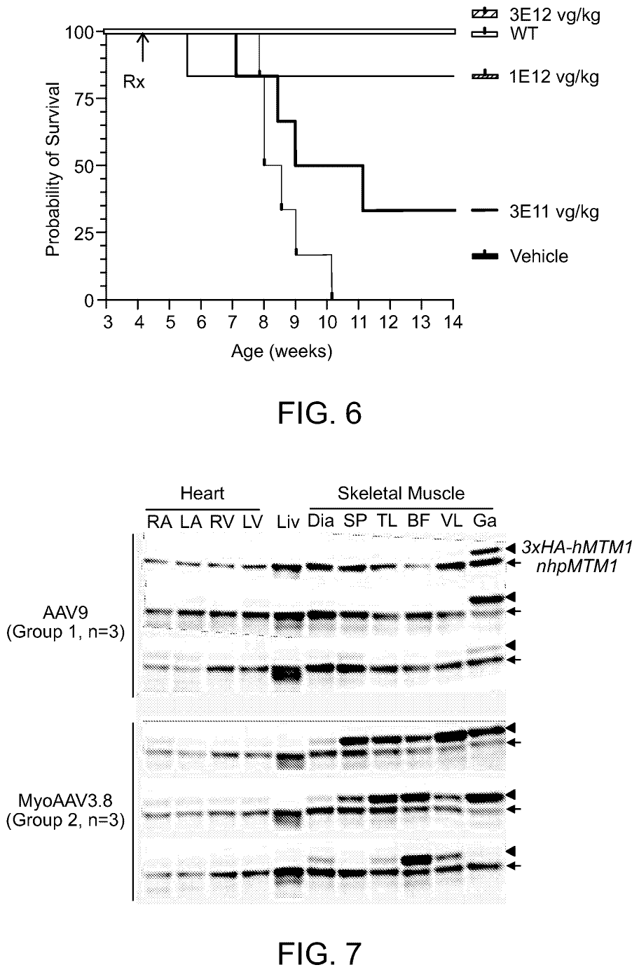

FIG. 6 is a graph of probability of survival following capsid and transgene administration in Mtm1 knockout (KO) mice. KT430 reversed XLMTM disease phenotype at very low doses (<3E12 vg/mg) in Mtm1 KO mice.

FIG. 7 is an image of MTM1 levels in non-human primate skeletal muscle following capsid and transgene administration. KT430 produced supra-physiological MTM1 levels in NHP skeletal muscle at 1E13 vg/kg.

### 6^{th} Generation Capsids

Additional modifications to AAV capsids enabled directed evolution of liver-de-targeted and muscle-tropic capsid variants.

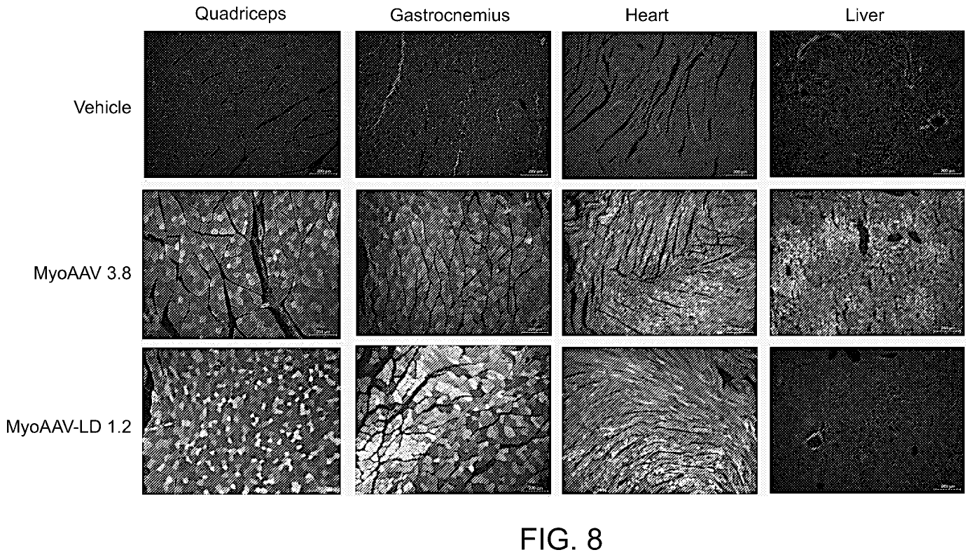

FIG. 8 shows images of fluorescence in tissues following administration of capsids of the invention.

Novel capsids (termed “6^{th} generation”) Myo-AAV-LD capsids were generated. Barcoded transgenes (CBh-hFXN) were packaged into different novel capsid variants. The pool of capsid variants were injected into 4 Cyno Macaques and expression of transgene from each capsid variant was quantified by sequencing the barcodes 4 weeks after injection.

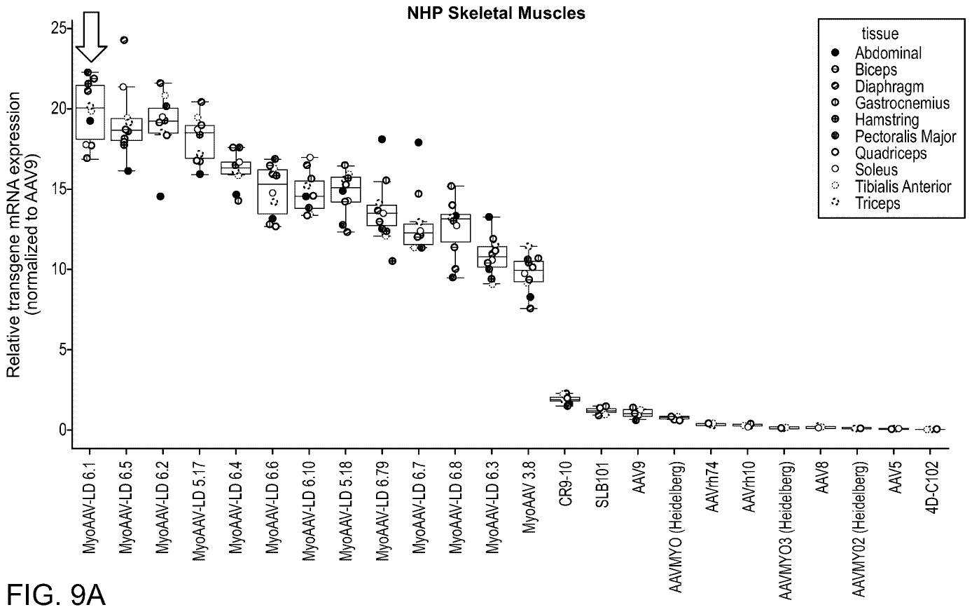

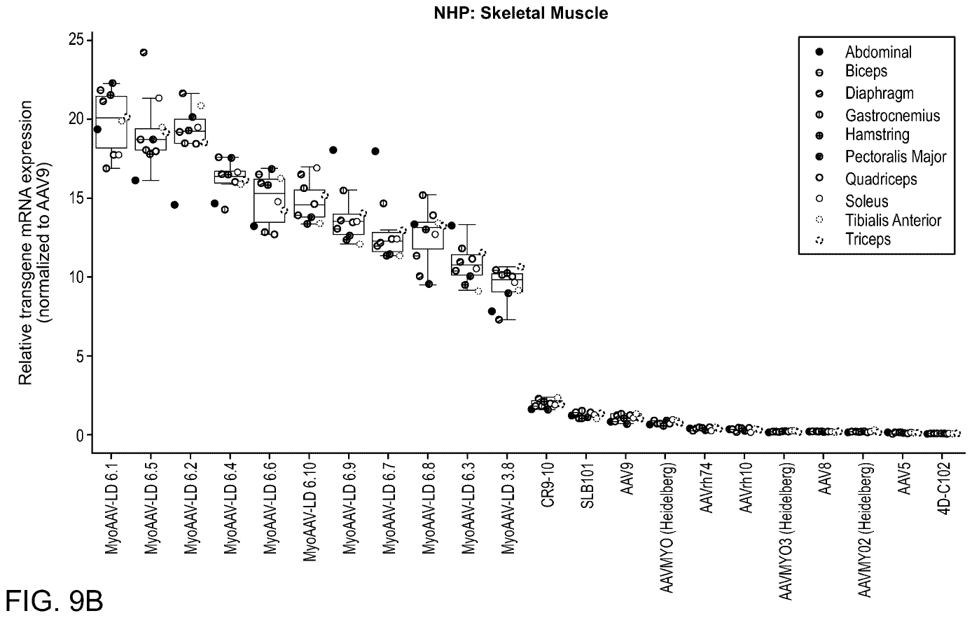

FIG. 9A-B show mRNA transgene expression levels in NHP skeletal muscle following administration of capsids of the invention.

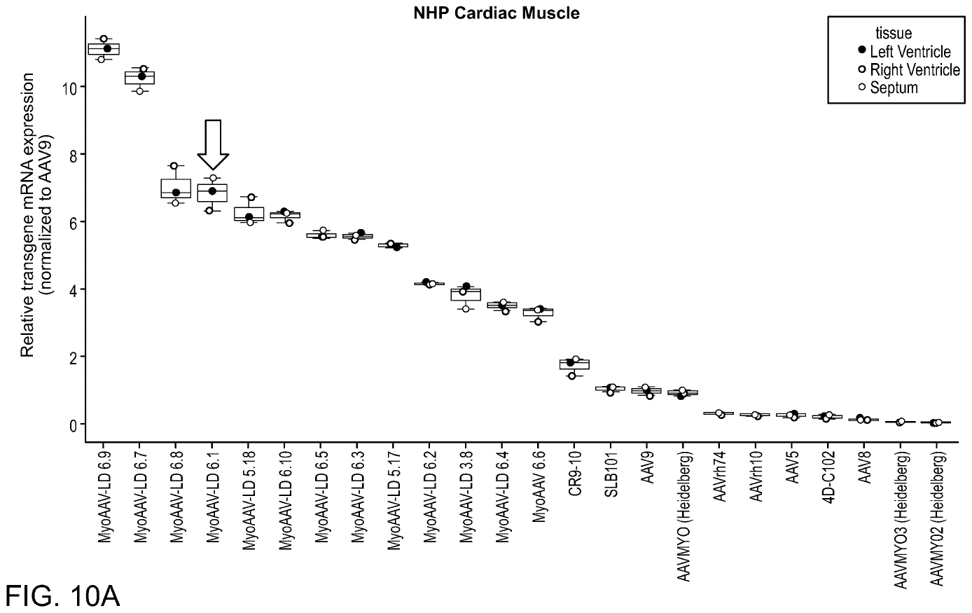

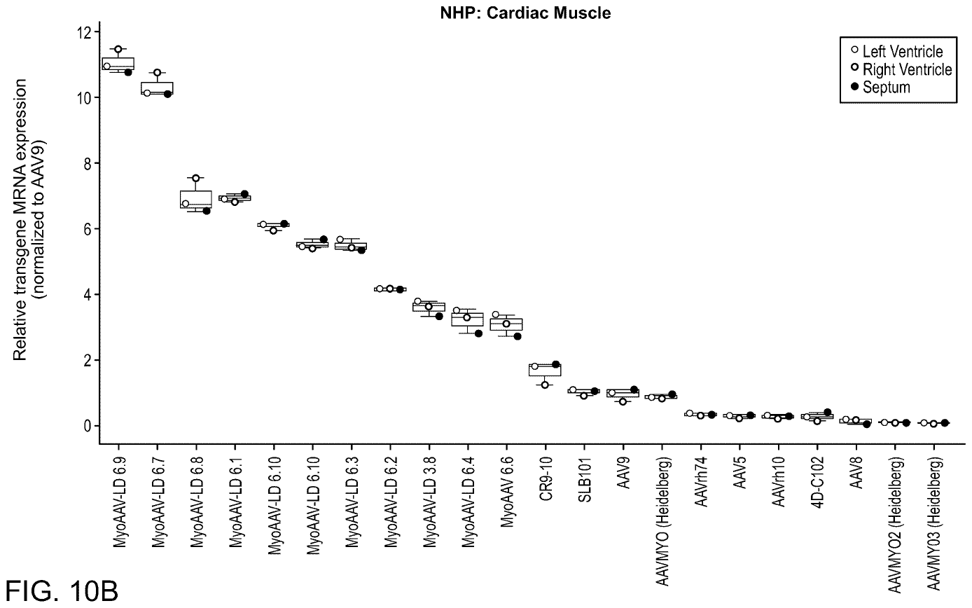

FIG. 10A-B show mRNA transgene expression levels in NHP cardiac muscle following administration of capsids of the invention.

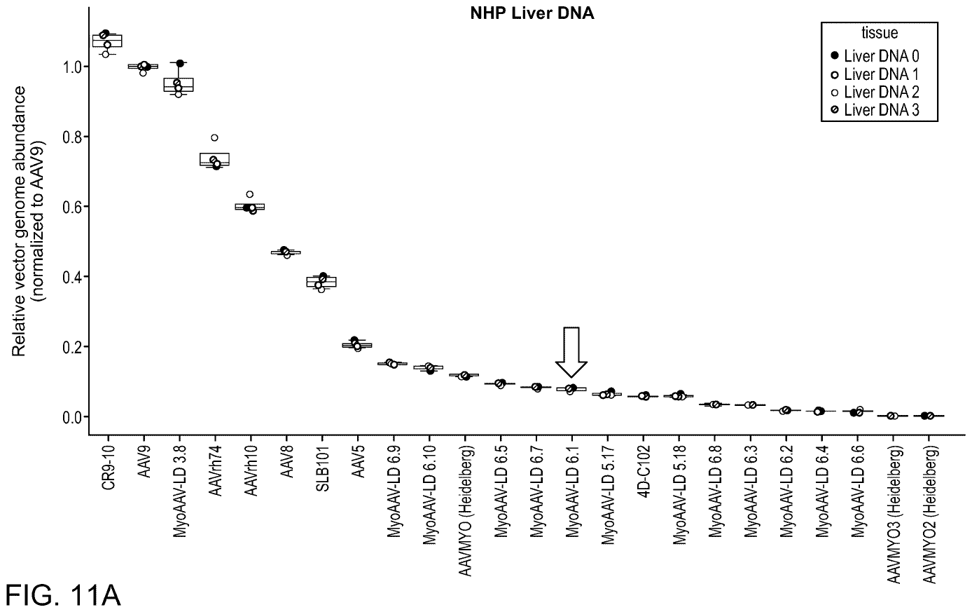

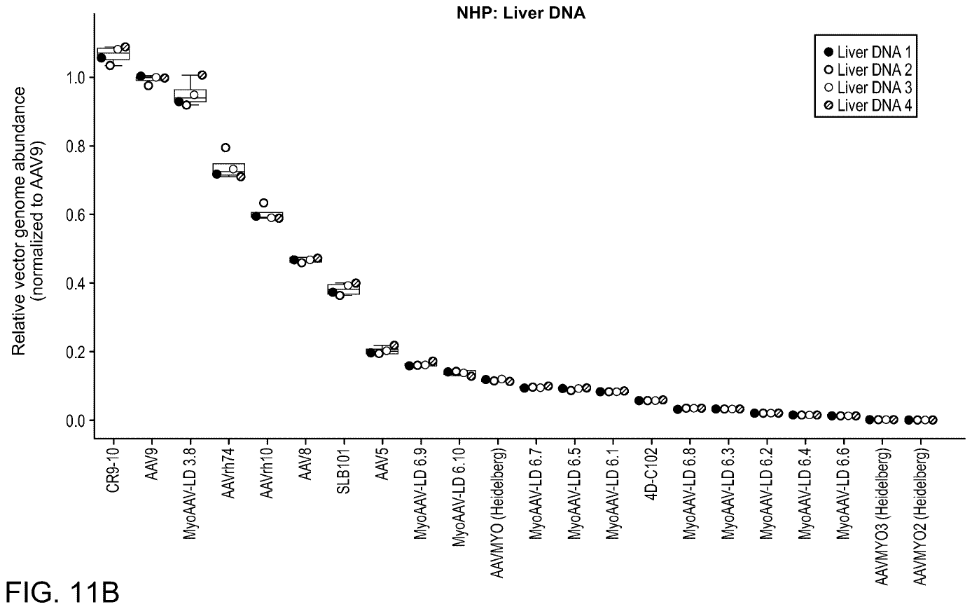

FIG. 11A-B show mRNA transgene expression levels in NHP liver following administration of capsids of the invention.

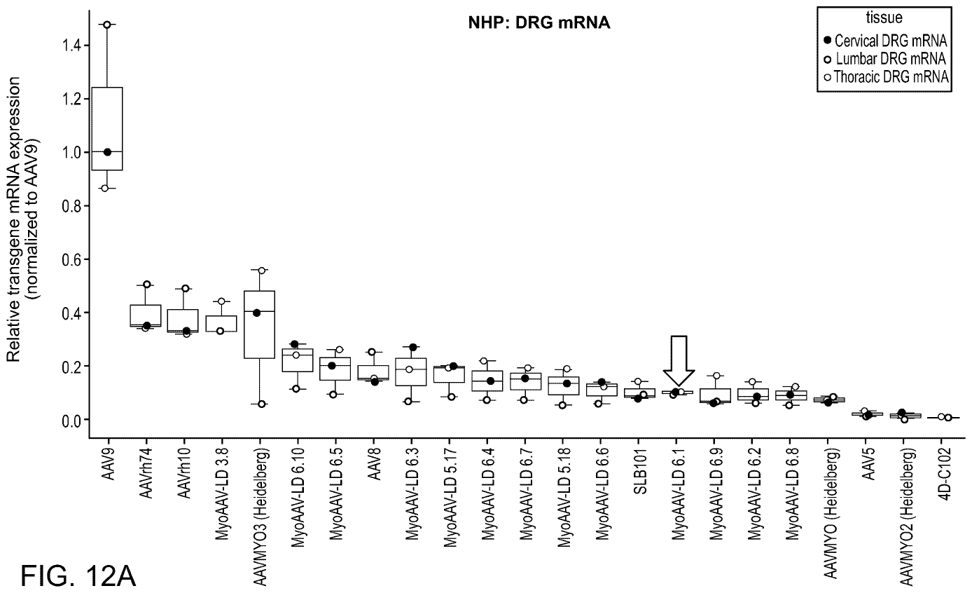

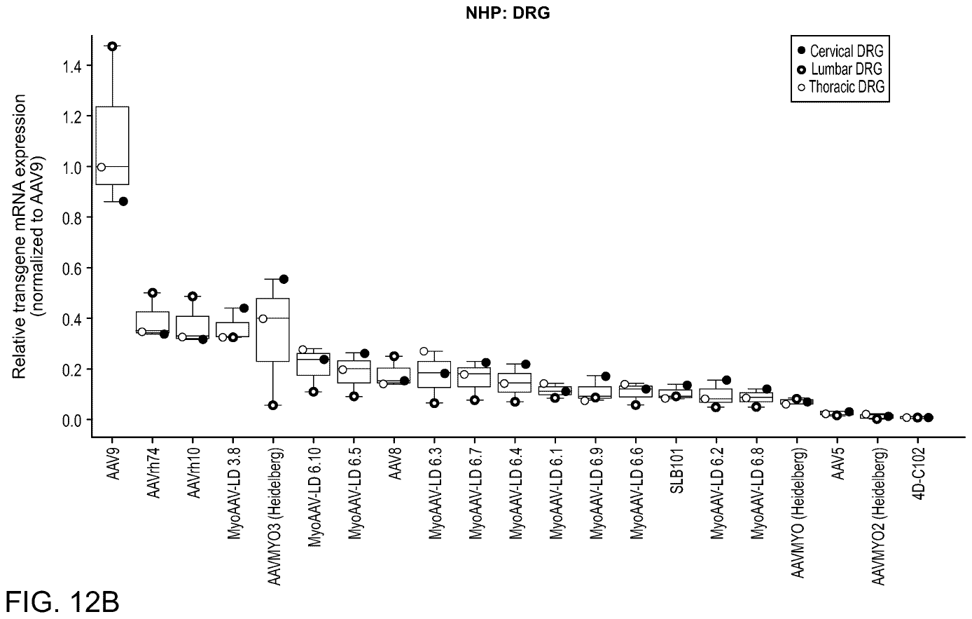

FIG. 12A-B show mRNA transgene expression levels in NHP dorsal root ganglia (DRG) following administration of capsids of the invention.

Capsids of the invention outperform in transducing NHL skeletal muscle versus naturally occurring and previously engineered capsids.

Capsids of the invention outperform in transducing NHL cardiac muscle versus naturally occurring and previously engineered capsids.

Capsids of the invention outperform were de-targeted from the liver at the DNA level in NHPs compared to naturally occurring capsids.

Capsids of the invention outperform were de-targeted from DRG in NHPs compared to naturally occurring capsids.

The top skeletal muscle tropic liver de-targeted MyoAAV-LD capsid, MyoAAV-LD 6.1, transduced NHP skeletal muscles ˜20 times more effectively and was ˜10 times de-targeted from the liver compared to AAV9.

The top cardiotropic liver de-targeted MyoAAV-LD capsid, MyoAAV-LD 6.9, transduced NHP cardiac muscle ˜11 times more effectively and was ˜6 times de-targeted from the liver compared to AAV9.

### DISCUSSION

Coevolution of liver de-targeting and muscle targeting features resulted in the development of MyoAAV-LD capsids in NHPs. The top skeletal muscle tropic liver de-targeted MyoAAV-LD capsid, MyoAAV-LD 6.1, transduced NHP skeletal muscles ˜20 times more effectively and was ˜10 times de-targeted from the liver compared to AAV9.

The top cardiotropic liver de-targeted MyoAAV-LD capsid, MyoAAV-LD 6.9, transduced NHP cardiac muscle ˜11 times more effectively and was ˜6 times de-targeted from the liver compared to AAV9.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

1.-23. (canceled)

24. An engineered adeno-associated virus (AAV) serotype 9 (AAV9) capsid protein comprising the amino acid sequence X₁NX₂X₃X₄RGDRX₅X₆L, wherein, relative to a wild-type AAV9 capsid protein,
X₁ is located at amino acid 451 and is any amino acid;
X₂ is located at amino acid 453 and is any amino acid;
X₃ is located at amino acid 454 and is any amino acid;
X₄ is located at amino acid 455 and is any amino acid; and
RGDRX₅X₆L is inserted after amino acid 455 and X₅-X₆ are any amino acid.

25. The engineered AAV9 capsid protein of claim 24, wherein:
X₁ is A, I, F, G, H, L, M, Q, S, T, or V;
X₂ is A, G, S, T, or Y;
X₃ is S, N, G, or P;
X₄ is A, G, H, I, M, S, T, or V;
X₅ is A, G, or Q; and
X₆ is A, I, L, M, N, S, or Y.

26. The engineered AAV9 capsid protein of claim 24, wherein:
X₁ is A, I, L, M, S, or V;
X₂ is A, G, S, T, or Y;
X₃ is S, N, G, or P;
X₄ is A, G, M, S, T, or V;
X₅ is A, G, or Q; and
X₆ is A, S, or Y.

27. The engineered AAV9 capsid protein of claim 24, wherein X₁NX₂X₃X₄ comprises an amino acid sequence of any one of one SEQ ID NOs: 801-1599, and RGDRX₅X₆L comprises an amino acid sequence of any one of SEQ ID NOs: 1600-2398.

28. The engineered AAV9 capsid protein of claim 24, wherein X₁NX₂X₃X₄ and RGDRX₅X₆L each correspond to the sequence of the 5 amino acid substitution and the 7 amino acid insert from a single row in a single column of Table 1b.

29. The engineered AAV9 capsid protein of claim 24, wherein the engineered AAV9 capsid protein further comprises a deletion of amino acid G267 relative to a wild-type AAV9.

30. The engineered AAV9 capsid protein of claim 24, wherein the engineered AAV9 capsid protein comprises the amino acid sequence X₁NX₂SX₄RGDRQX₆L, and wherein, relative to a wild-type AAV9 capsid protein,
X₁ is L, S, or V substituted for I at amino acid 451;
X₂ is G or is A or Y substituted for G at amino acid 451;
X₄ is T, I, M, or V substituted for G at amino acid 455; and
RGDRQX₆L is inserted after amino acid 455 and X₆ is Y, A, L, or S.

31. The engineered AAV9 capsid protein of claim 30, wherein the engineered AAV9 capsid protein further comprises a deletion of amino acid G267 relative to a wild-type AAV9.

32. The engineered AAV9 capsid protein of claim 30, wherein the engineered AAV9 capsid protein comprises the amino acid sequence LNASIRGDRQLL (SEQ ID NO: 191), wherein, relative to a wild-type AAV9 capsid protein, L is substituted for I at amino acid 451, A is substituted for G at amino acid 453, I is substituted for G at amino acid 455, and RGDRQLL (SEQ ID NO: 1789) is inserted after amino acid 455 in reference to a wild-type AAV9 capsid protein.

33. The engineered AAV9 capsid protein of claim 32, wherein the engineered AAV9 capsid protein further comprises a deletion of amino acid G267 relative to a wild-type AAV9.

34. The engineered AAV9 capsid protein of claim 30, wherein the engineered AAV9 capsid protein comprises the amino acid sequence LNASMRGDRQAL (SEQ ID NO: 198), wherein, relative to a wild-type AAV9 capsid protein, L is substituted for I at amino acid 451, A is substituted for G at amino acid 453, M is substituted for G at amino acid 455, and RGDRQAL (SEQ ID NO: 1977) is inserted after amino acid 455 in reference to a wild-type AAV9 capsid protein.

35. The engineered AAV9 capsid protein of claim 34, wherein the engineered AAV9 capsid protein further comprises a deletion of amino acid G267 relative to a wild-type AAV9.

36. The engineered AAV9 capsid protein of claim 30, wherein the engineered AAV9 capsid protein comprises the amino acid sequence LNASMRGDRQSL (SEQ ID NO: 199), wherein, relative to a wild-type AAV9 capsid protein, L is substituted for I at amino acid 451, A is substituted for G at amino acid 453, M is substituted for G at amino acid 455, and RGDRQSL (SEQ ID NO: 1797) is inserted after amino acid 455 in reference to a wild-type AAV9 capsid protein.

37. The engineered AAV9 capsid protein of claim 36, wherein the engineered AAV9 capsid protein further comprises a deletion of amino acid G267 relative to a wild-type AAV9.

38. The engineered AAV9 capsid protein of claim 30, wherein the engineered AAV9 capsid protein comprises the amino acid sequence LNASTRGDRQYL (SEQ ID NO: 215), wherein, relative to a wild-type AAV9 capsid protein, L is substituted for I at amino acid 451, A is substituted for G at amino acid 453, T is substituted for G at amino acid 455, and RGDRQYL (SEQ ID NO: 1813) is inserted after amino acid 455 in reference to a wild-type AAV9 capsid protein.

39. The engineered AAV9 capsid protein of claim 38, wherein the engineered AAV9 capsid protein further comprises a deletion of amino acid G267 relative to a wild-type AAV9.

40. The engineered AAV9 capsid protein of claim 30, wherein the engineered AAV9 capsid protein comprises the amino acid sequence LNGSTRGDRQYL (SEQ ID NO: 256), wherein, relative to a wild-type AAV9 capsid protein, L is substituted for I at amino acid 451, T is substituted for G at amino acid 455, and RGDRQYL (SEQ ID NO: 1813) is inserted after amino acid 455 in reference to a wild-type AAV9 capsid protein.

41. The engineered AAV9 capsid protein of claim 40, wherein the engineered AAV9 capsid protein further comprises a deletion of amino acid G267 relative to a wild-type AAV9.

42. The engineered AAV9 capsid protein of claim 30, wherein the engineered AAV9 capsid protein comprises the amino acid sequence LNYSTRGDRQAL (SEQ ID NO: 379), wherein, relative to a wild-type AAV9 capsid protein, L is substituted for I at amino acid 451, Y is substituted for G at amino acid 453, T is substituted for G at amino acid 455, and RGDRQAL (SEQ ID NO: 1977) is inserted after amino acid 455 in reference to a wild-type AAV9 capsid protein.

43. The engineered AAV9 capsid protein of claim 42, wherein the engineered AAV9 capsid protein further comprises a deletion of amino acid G267 relative to a wild-type AAV9.

44. The engineered AAV9 capsid protein of claim 30, wherein the engineered AAV9 capsid protein comprises the amino acid sequence SNASTRGDRQYL (SEQ ID NO: 544), wherein, relative to a wild-type AAV9 capsid protein, S is substituted for I at amino acid 451, A is substituted for G at amino acid 453, T is substituted for G at amino acid 455, and RGDRQYL (SEQ ID NO: 1813) is inserted after amino acid 455 in reference to a wild-type AAV9 capsid protein.

45. The engineered AAV9 capsid protein of claim 44, wherein the engineered AAV9 capsid protein further comprises a deletion of amino acid G267 relative to a wild-type AAV9.

46. The engineered AAV9 capsid protein of claim 30, wherein the engineered AAV9 capsid protein comprises the amino acid sequence SNASVRGDRQAL (SEQ ID NO: 550), wherein, relative to a wild-type AAV9 capsid protein, S is substituted for I at amino acid 451, A is substituted for G at amino acid 453, Vis substituted for G at amino acid 455, and RGDRQAL (SEQ ID NO: 1977) is inserted after amino acid 455 in reference to a wild-type AAV9 capsid protein.

47. The engineered AAV9 capsid protein of claim 46, wherein the engineered AAV9 capsid protein further comprises a deletion of amino acid G267 relative to a wild-type AAV9.

48. The engineered AAV9 capsid protein of claim 30, wherein the engineered AAV9 capsid protein comprises the amino acid sequence VNGSTRGDRQYL (SEQ ID NO: 748), wherein, relative to a wild-type AAV9 capsid protein, Vis substituted for I at amino acid 451, T is substituted for G at amino acid 455, and RGDRQYL (SEQ ID NO: 1813) is inserted after amino acid 455 in reference to a wild-type AAV9 capsid protein.

49. The engineered AAV9 capsid protein of claim 48, wherein the engineered AAV9 capsid protein further comprises a deletion of amino acid G267 relative to a wild-type AAV9.

50. An AAV vector comprising the engineered AAV9 capsid protein of claim 24 and a transgene.

51. The AAV vector of claim 49, wherein the AAV vector exhibits increased muscle tropism as compared to a wild-type AAV vector.

52. The AAV vector of claim 49, wherein the AAV vector exhibits reduced liver tropism as compared to a wild-type AAV vector.

53. A method of treating a genetic disease comprising administering the AAV vector of claim 49.
